(19) United States
(12) Patent Application Publication
(10) Pub. No.: US 2026/0209225 A1
(43) Pub. Date: Jul. 23, 2026

(54) PYRROLOPYRIDINE AND PYRROLOPYRIDAZINE BASED COMPOUNDS AND USE THEREOF AS PKR INHIBITORS

(71) Applicant: PROTEKT THERAPEUTICS LTD., Ness-Ziona (IL)

(72) Inventors: Yotam NISEMBLAT, Raanana (IL); Dalia PINKERT, Kfar-Sava (IL)

(21) Appl. No.: 19/140,568

(22) PCT Filed: Dec. 21, 2023
(86) PCT No.: PCT/IL2023/051297

§ 371 Date: Jun. 18, 2025

Related U.S. Application Data
(60) Provisional application No. 63/434,853, filed on Dec. 22, 2022.

(51) Int. Cl. C07D 471/04; A61K 31/437; A61K 31/444; A61K 31/506; C07D 519/00

(52) U.S. Cl. CPC C07D 471/04; A61K 31/437; A61K 31/444; A61K 31/506; C07D 519/00

(57) ABSTRACT

The present invention is directed to a composition and a method for use thereof, such as for the treatment and prevention of a neurological disorder, a neuroinflammatory disorder, a cognitive disorder or cancer in a subject.

### CROSS-REFERENCE TO RELATED APPLICATIONS

This application is a PCT International Application claiming the benefit of priority of U.S. Provisional Patent Application No. 63/434,853, filed Dec. 22, 2022, which is hereby incorporated by reference in its entirety.

### FIELD OF THE INVENTION

The invention relates generally to the field of compositions comprising one or more pyrrolopyridine or pyrrolopyridazine derivatives and is directed to methods of using the same such as for treating of a Protein kinase-R (PKR) related disease.

### BACKGROUND

Neurodegenerative disorders (ND) constitute a class of diseases that express characteristic misfolded proteins that aggregate and induce neuronal toxicity and death. Various NDs are known, including inter alia multiple sclerosis, Alzheimer's disease, dementia, Parkinson's disease, Huntington's disease, Down syndrome, Amyotrophic lateral sclerosis (ALS), and prion disease. Currently there is no reliable therapeutic avenue that can substantially delay or stop the progression of the disease. In this context, there is a need to develop therapeutics and drug targets that can prevent or delay pathogenesis in neurodegenerative diseases.

The foregoing examples of the related art and limitations related therewith are intended to be illustrative and not exclusive. Other limitations of the related art will become apparent to those of skill in the art upon a reading of the specification.

### SUMMARY

The following embodiments and aspects thereof are described and illustrated in conjunction with systems, tools and methods which are meant to be exemplary and illustrative, not limiting in scope.

In one aspect of the invention, there is a compound or a salt thereof, wherein the compound is represented by or comprises Formula I:

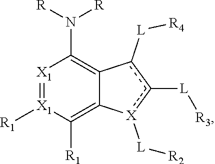

or Formula IB:

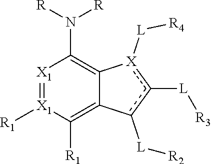

wherein:
[symbol] represents a single or a double bond;
each L is absent or independently comprises an optionally substituted C₁-C₆ alkyl, N, NH, S, and O, —S(O)₂—, —C(O)—, —(C₁-C₆)—O—(C₁-C₆)₀₋₁—, —(C₁-C₆)—S—(C₁-C₆)₀₋₁—, —(C₁-C₆)—NH—(C₁-C₆)₀₋₁—, or any combination thereof;
each X₁ is independently selected from the group consisting of CH, C, or N;
at least one X₁ is N;
X comprises S, O, N or NH;
each R₁ independently represents hydrogen, or a substituent comprising halogen, —NO₂, —CN, —OH, —(C═O)NH2, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, optionally substituted aryl, optionally substituted heteroaryl, optionally substituted heterocyclyl, and optionally substituted C₃-C₈ cycloalkyl or a combination thereof, wherein at least one R₁ represents the substituent;
each of R₂, R₃ and R₄ independently represents hydrogen, or is selected from the group comprising halogen, —NO₂, —CN, —OH, —(C═O)NH2, —CONR₂, —CO₂R, —SO₂R, optionally substituted C₁-C₆ alkyl, —NH₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof;
wherein each R independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl or a combination thereof.

In one embodiment, the compound is represented by or comprises Formula IIA:

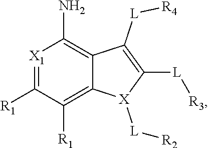

### or Formula IIB:

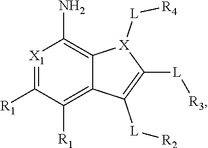

In one embodiment, the compound is represented by or comprises Formula III:

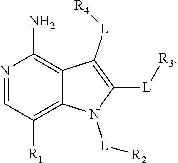

In one embodiment, each R₁ independently represents hydrogen, or a substituent comprising halogen, —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, or a combination thereof, wherein at least one R₁ represents the substituent.

In one embodiment, the compound is represented by or comprises Formula IV:

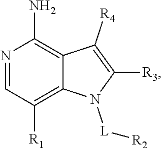

wherein R₁ is selected form the group consisting of halogen, —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, —CO₂R, —OCOR, —OCOR, —OC(═O)OR, —OC(═O)NR, —OC(═S)OR, —OC(═S)NR, or a combination thereof.

In one embodiment, R₂ represents hydrogen, or a substituent comprising C₁-C₆ alkyl, C₁-C₆ haloalkyl, C₁-C₆ alkyl-NR₂, C₁-C₆ alkyl-OR, C₁-C₆ alkyl-SR, C₁-C₆ alkyl-Z, —Z, optionally substituted C₃-C₈ cycloalkyl, optionally substituted (C₅-C₆) heteroaryl, optionally substituted aryl, optionally substituted bicyclic heteroaryl, optionally substituted bicyclic aryl, optionally substituted bicyclic heterocyclyl, optionally substituted bicyclic cycloalkyl, or a combination thereof; wherein Z is selected from the group consisting of —CO₂R, CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —CO₂R, CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, or a combination thereof.

In one embodiment, R₃ and R₄ each independently represents hydrogen, or a substituent comprising halogen, unsaturated C₁-C₆ alkyl, C₁-C₆ haloalkyl, optionally substituted alkaryl, optionally substituted heteroaryl, optionally substituted aryl, optionally substituted bicyclic heteroaryl, optionally substituted bicyclic heterocyclyl, optionally substituted bicyclic cycloalkyl, or a combination thereof.

In one embodiment, the compound is represented by or comprises Formula V:

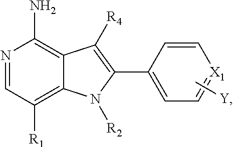

### or by Formula VI:

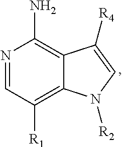

wherein Y is absent or is selected form the group consisting of (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, or a combination thereof; and wherein R₄ is selected from the group comprising any one of hydrogen, halogen,

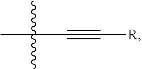

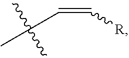

C₁-C₆ haloalkyl, C₁-C₆ alkyl, heterocyclyl optionally substituted by Y, and an aryl optionally substituted by Y; wherein the aryl is selected from the group consisting of pyridine, phenyl, pyrimidine, pyridazine, pyrazine, triazine, pyrazole, pyrrole, triazole, imidazole, indole, iso-indole, isoindolin-1-one indazole, benzimidazole, azaindazole, purine, azaindole, and indoline or a combination thereof.

In one embodiment, R₂ represents hydrogen, or a substituent comprising C₁-C₆ alkyl, C₁-C₆ haloalkyl, C₁-C₆ alkyl-NR₂, C₁-C₆ alkyl-OR, C₁-C₆ alkyl-CONR₂, C₁-C₆ alkyl-COOR, C₁-C₆ alkyl-CNNR₂, C₁-C₆ alkyl-CSNR₂, C₃-C₆ cycloalkyl optionally substituted by Y, and a C₆ aryl optionally comprising a heteroatom selected from N and O, and optionally substituted by Y; wherein the C₆ aryl is selected from the group consisting of pyridine, phenyl, pyrimidine, pyridazine, pyrazine, triazine or a combination thereof.

In one embodiment, R₂ represents a substituent comprising any one of (i) C₁-C₆ alkyl, or C₁-C₆ alkyl-CONR₂; and (ii) a C₅-C₆ cycloalkyl optionally substituted by halogen, —NO₂, —NH₂, —NH(C₁-C₆ alkyl), —N(C₁-C₆ alkyl)₂, hydroxy(C₁-C₆ alkyl), —CN, —OH, —(C═O)NH₂, —CON(C₁-C₆ alkyl)₂, —CON(C₁-C₆ alkyl)₂, —CONH(C₁-C₆ alkyl), C₁-C₆ haloalkyl, and —SO₂(C₁-C₆ alkyl), or a combination thereof, wherein C₅-C₆ cycloalkyl is selected form the group consisting of cyclopentane, cyclohexane, pyridine and phenyl or a combination thereof.

In one embodiment, R₂ represents a substituent comprising any one of

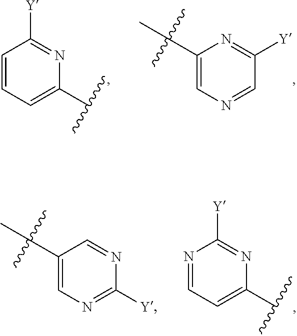

wherein Y′ is selected form the group consisting of hydrogen, halo, —NO₂, —NH₂, —OMe, —(C═O)NH₂, —SO₂(C₁-C₆), and C₁-C₆ haloalkyl, or a combination thereof.

In one embodiment, R₄ comprises or is represented by

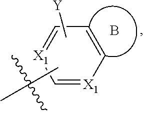

wherein B comprises any one of:

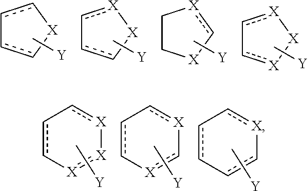

or is absent, and wherein X1 is selected from C, CH, and N.

In one embodiment, R₄ is represented by any one of:

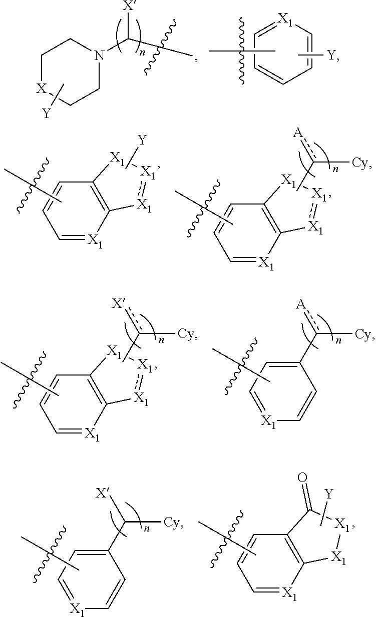

wherein A comprises a heteroatom selected from N, O, S and P; wherein X′ comprises halogen, hydroxy, —CN, nitro, mercapto, amino, C₁-C₆ alkyl, C₁-C₆ haloalkyl, C₁-C₆ alkoxy, C₁-C₆ haloalkoxy, hydroxy(C₁-C₆ alkyl), amino(C₁-C₆ alkyl), optionally substituted C₃-C₈ cycloalkyl, —CO₂R, —CON(R′)₀₋₂, —CNNR′₂, —CSNR′₂, —CONH—OH, —CONH—NH₂, —NHCOR, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —CO₂H, or a combination thereof, wherein X1 is selected from C, CH, and N; and wherein Cy comprises optionally substituted (C₅-C₆)heterocyclyl, optionally substituted C₁-C₆ alkyl-(C₅-C₆)heterocyclyl, optionally substituted alkaryl, optionally substituted (C₅-C₆)heteroaryl, optionally substituted aryl, optionally substituted bicyclic heteroaryl, optionally substituted bicyclic heterocyclyl, optionally substituted cycloalkyl, optionally substituted bicyclic cycloalkyl or a combination thereof, and wherein n is between 0 and 5.

In one embodiment, R₄ is an aryl selected from the group consisting of pyridine, phenyl, pyrimidine, pyridazine, pyrazine, triazine, pyrazole, indole, azaindole, indoline and isoindolin-1-one or a combination thereof, wherein the aryl is optionally substituted by any one of Y,

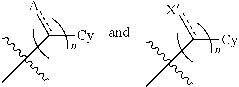

or a combination thereof.

In one embodiment, R₁ is selected from the group consisting of halogen, —NO₂, —CN, —(C═O)NH₂, —CONH(C₁-C₆ alkyl)₂, C₁-C₆ haloalkyl, —CO₂(C₁-C₆ alkyl), —CO₂H, —SO₂H, —SO₂(C₁-C₆ alkyl), or a combination thereof.

In one embodiment, the compound is stable in an aqueous solution for at least 1 hour.

In another aspect, there is provided a compound represented by or comprising Formula IA:

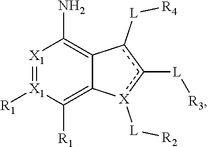

### or Formula IB:

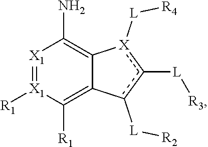

wherein: [symbol] represents a single or a double bond; each L is absent or independently comprises an optionally substituted C₁-C₆ alkyl, N, NH, S, and O, —S(O)₂—, —C(O)—, —(C₁-C₆)—O—(C₁-C₆)₀₋₁—, —(C₁-C₆)—S—(C₁-C₆)₀₋₁—, —(C₁-C₆)—NH—(C₁-C₆)₀₋₁—, or any combination thereof; each X₁ is independently selected from the group consisting of CH, C, or N, and at least one X₁ is N; X comprises S, O, N or NH; each R₁ independently represents hydrogen, or a substituent comprising halogen, —NO₂, —CN, —OH, —(C═O)NH₂, HCONH—, oxo, carbonyl, amino, imino, thioxo, —OR, —(C═O)NH₂, —CO₂R, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NR₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), C₁-C₆ mercaptoalkyl, —CONH(C₁-C₆ alkyl), —CON(C₁-C₆ alkyl)₂, —CO₂H, —CO₂R, —OCOR, —OC(═O)OR, —OC(═O)NR, —OC(═S)OR, —OC(═S)NR, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, wherein at least one R₁ represents the substituent; any of R₂, R₃ and R₄ is independently H or represents a substituent comprising halogen, —NO₂, —CN, —OH, —OR, —SR, —(C═O)NH2, —CONR₂, —CO₂R, —SO₂R, oxo, carbonyl, amino, C₁-C₆ mercaptoalkyl, optionally substituted C₁-C₆ alkyl, optionally substituted C₁-C₆ heteroalkyl, imino, thioxo, carbonyl, aminoalkyl, fluoroalkyl, optionally substituted alkyl-aryl/heteroaryl, —O—C₁-C₆ haloalkyl, —O—C(R)₂—C₁-C₆ haloalkyl, —NR₂, -M-W, an optionally substituted C₃-C₈ saturated/unsaturated heterocyclyl, optionally substituted C₃-C₈ saturated/unsaturated cycloalkyl, optionally substituted C₂-C₆ alkenyl, optionally substituted C₂-C₆ alkynyl, —C(═O)—R, —S—C₁-C₆ haloalkyl, —NH—C₁-C₆ haloalkyl, —NR₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, or any of R₂, R₃ and R₄ is absent; wherein at least one of R₂ and R₃ is not H or absent; wherein M is selected from —O—, —S—, —NH—, —C(═O)—, —CONR—, —CNNR—, —CSNR—, —CONH—O—, —CONH—NH—, —NHCO—, —NHCSR—, —NHCN—, —NC(═O)O—, —NC(═O)N—, —NC(═S)O—, —NC(═S)N—, —SO₂—, —SO—, —SO₂O—, —SO₂NR—, —NHNR—, —CO₂—, —OCO—, —OC(═O)O—, —OC(═O)N—, —OC(═S)O— and —OC(═S)N—; wherein W represents an optionally substituted C₃-C₈ cycloalkyl optionally substituted C₃-C₈ heterocyclyl optionally substituted aryl, optionally substituted alkyl-aryl/heteroaryl, or optionally substituted heteroaryl; wherein each R independently represents hydrogen, or is selected from the group comprising optionally substituted C₁-C₁₀ alkyl, optionally substituted C₁-C₁₀ alkenyl/alkynyl, hydroxy, amino, —NH₂, —NR₂, —NH(C₁-C₆ alkyl), —N(C₁-C₆ alkyl)₂, C₁-C₆ alkoxy, —OR, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, optionally substituted C₃-C₁₀ cycloalkyl, optionally substituted C₁-C₁₀ alkaryl (e.g. benzyl), optionally substituted C₁-C₁₀ alkyl-C₃-C₁₀ cycloalkyl, optionally substituted C₃-C₁₀ heterocyclyl, optionally substituted heteroaryl, optionally substituted aryl, or a combination thereof, or a salt thereof, wherein the compound comprises any tautomer, any isotope, or any salt thereof.

In one embodiment, the compound is represented by or comprises Formula III:

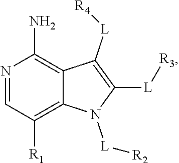

### or Formula IIIA

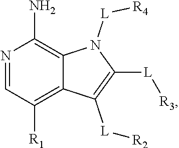

wherein R₁ is selected from halo, cyano, and formamidyl (i.e., —(C═O)NH₂ or —NH(C═O)H); and wherein R₂ represents H or a substituent comprising C₁-C₆ haloalkyl, C₁-C₆ fluoroalkyl, —NR₂, C₁-C₆ alkyl-NR₂, C₁-C₆ alkyl-OR, C₁-C₆ alkyl-CONR₂, C₁-C₆ alkyl-COOR, C₁-C₆ alkyl-CNNR₂, C₁-C₆ alkyl-CSNR₂, optionally substituted C₅ or C₆ heteroaryl; and if R2 is H then R3 is not H.

In one embodiment, the compound is represented by or comprises Formula IV:

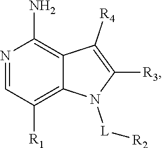

wherein R₁ is selected is selected from halo (e.g. fluoro), cyano, and formamidyl; wherein R₂ represents H or a substituent comprising C₁-C₆ fluoroalkyl, —NR₂ or optionally substituted C₅ or C₆ heteroaryl; and if R2 is H then R3 is not H.

In one embodiment, R3 is H, and R₂ represents any of:

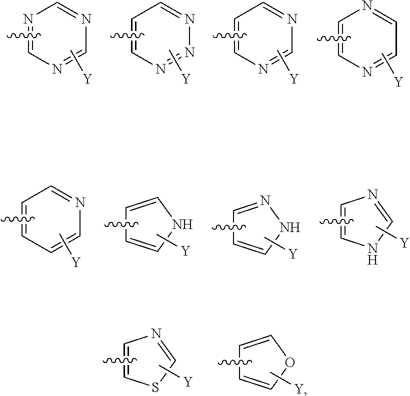

wherein Y is hydrogen or represents one or more substituents, each independently selected from halo, C₁-C₆ fluoroalkyl, —NO₂, —NH₂, —O(C₁-C₆ alkyl), —OMe, —NH(C₁-C₆ alkyl), —N(C₁-C₆ alkyl)₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, —(C₁-C₆)—OH, or a combination thereof. The compound of the invention, wherein R₃ is selected from halogen, —NO₂, —CN, —OH, —OR, —(C═O)NH₂, —CONR₂, —SR, —CO₂R, —SO₂R, optionally substituted C₁-C₆ alkyl, amino, oxo, imino, thioxo, carbonyl, aminoalkyl, —NH₂, —NH(C₁-C₆ alkyl), hydroxy(C₁-C₆ alkyl), C₁-C₆ haloalkyl, C₁-C₆ mercaptoalkyl, C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof.

In one embodiment, the compound is represented by or comprises Formula V:

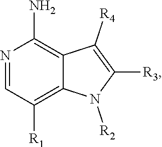

### or by Formula VA:

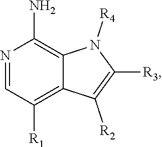

wherein: R₁ is selected from halo, cyano, and formamidyl; R₃ is H or is selected from (i) halogen, —NO₂, —CN, —OH, —OR, —(C═O)NH₂, —CONR₂, —SR, —CO₂R, —SO₂R, optionally substituted C₁-C₆ alkyl, amino, oxo, imino, thioxo, carbonyl, aminoalkyl, —NH₂, —NH(C₁-C₆ alkyl), hydroxy(C₁-C₆ alkyl), C₁-C₆ haloalkyl, C₁-C₆ mercaptoalkyl, C₁-C₆ haloalkyl and (ii) optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; if R₃ is H or (i) then R₂ is selected from C₁-C₆ fluoroalkyl and a heteroaryl optionally substituted by one or more substituents, optionally wherein the substituents are interconnected so as to form a 5-, or 6-membered aliphatic, unsaturated or aromatic ring; if R₃ or R₄ is H then R₂ is not H; if R₃ is (ii) then R₂ is C₁-C₆ fluoroalkyl.

In one embodiment, R₃ is H, R₂ is selected from C₁-C₆ fluoroalkyl and a heteroaromatic ring optionally substituted by one or more substituents, optionally wherein the substituents are interconnected so as to form a 5-, or 6-membered aliphatic, unsaturated or aromatic ring.

In one embodiment, R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

In one embodiment, R₂ is a C1-C3 fluoroalkyl, or a 5-6 membered heteroaromatic ring substituted by one or more substituent selected from —F and C1-C3 fluoroalkyl.

In one embodiment, the compound is represented by Formula VB:

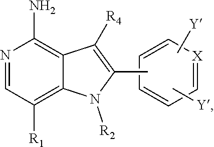

wherein X is N or CH; wherein each Y′ is independently hydrogen or one or more substituent selected from optionally substituted C₁-C₆ alkyl, —OR, —NR₂, —O(C₁-C₆ alkyl), —OMe, —NH(C₁-C₆ alkyl), —N(C₁-C₆ alkyl)₂, —CN, —OH, —NHCOR, —NHNR₂, —NNR, C₁-C₆ haloalkyl, —(C₁-C₆)OH, or wherein both Y′ are interconnected so as to form a ring; and wherein R₂ is selected from H and C₁-C₆ fluoroalkyl; and wherein R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

In one embodiment, the compound is represented by Formula VI:

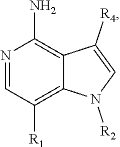

wherein R₁ is selected from cyano and formamidyl; R₂ is selected from C₁-C₆ haloalkyl, optionally substituted alkyl-aryl/heteroaryl, and a 5-6 membered heteroaromatic ring optionally substituted by at least one of —F and C₁-C₃ fluoroalkyl; R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

In one embodiment, R₂ is selected from —CH2F and a 5-6 membered heteroaromatic ring substituted by at least one of —F and C₁-C₃ fluoroalkyl.

In one embodiment, R₂ is selected from —CH2F and pyridine substituted by at least one of —F and C1-C3 fluoroalkyl; and wherein R₄ is selected from fluoroalkyl, halo, —OR, —C(═O)—R, substituted C₃-C₆ cycloalkyl, substituted C₃-C₆ heterocyclyl comprising an unsaturated bond and a substituted aryl.

In one embodiment, the compound is or comprises any one of compounds of Table 1 or FIG. 4.

In another aspect, there is a pharmaceutical composition, comprising the compound of the invention and a pharmaceutically acceptable carrier.

In one embodiment, the pharmaceutical composition is for use in the inhibition of protein kinase-R (PKR) cellular activity.

In one embodiment, the cell is a nerve cell, a glia cell or both.

In another aspect, there is a method for preventing or treating a disease or a disorder or ameliorating a condition associated with an activity of PKR in a subject, comprising administering to the subject the pharmaceutical composition of the invention, thereby (i) preventing or treating a disease or a disorder; or (ii) ameliorating the condition associated with an abnormal expression and/or activation of the PKR in a subject.

In one embodiment, the disease or the disorder comprises a neurodegenerative disorder, a neuroinflammatory disorder, a metabolic disease, a cardiovascular disease, a proliferative disease, or any combination thereof.

In one embodiment, the neurodegenerative disorder is selected from the group comprising: Alzheimer's disease, multiple sclerosis, dementia, Parkinson's disease, Huntington's disease, Down syndrome, Amyotrophic lateral sclerosis (ALS), frontotemporal dementia (FTD), Dystonia and prion disease or any combination thereof.

In one embodiment, preventing or treating comprises inhibiting the PKR function in the cell.

In one embodiment, administering is selected from oral administration, a topical administration, nasal administration, sublingual administration, buccal administration, a systemic administration, or any combination thereof.

In one embodiment, ameliorating comprises (i) improving cognitive function, or (ii) inhibiting cognitive dysfunction of the subject.

In addition to the exemplary aspects and embodiments described above, further aspects and embodiments will become apparent by study of the following detailed description.

### BRIEF DESCRIPTION OF THE DRAWINGS

FIG. 1 is a scheme presenting a non-limiting general synthetic pathway for some of the exemplary compounds of the invention.

FIG. 2 is a scheme presenting a non-limiting general synthetic pathway for some of the exemplary compounds of the invention.

FIG. 3 is a scheme presenting a non-limiting general synthetic pathway for some of the exemplary compounds of the invention.

FIG. 4 presents chemical structures of the example compounds of the invention.

### DETAILED DESCRIPTION

In one aspect of the invention disclosed herein, there is a compound represented by Formula I:

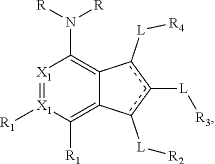

### or by Formula IB:

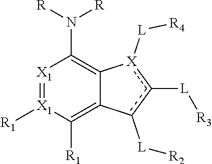

wherein [symbol] represents a single or a double bond;
each L is absent or independently comprises an optionally substituted C₁-C₆ alkyl, N, NH, S, and O, —S(O)₂—, —C(O)—, —(C₁-C₆)—O—(C₁-C₆)₀₋₁—, —(C₁-C₆)—S—(C₁-C₆)₀₋₁—, —(C₁-C₆)—NH—(C₁-C₆)₀₋₁—, or any combination thereof, each X₁ independently comprises CH, C, or N; X comprises S, O, N or NH; each R₁ independently represents hydrogen, or a substituent comprising halogen, —NO₂, —CN, —OH, —(C═O)NH₂, HCONH—, oxo, carbonyl, amino, imino, thioxo, —OR, —(C═O)NH₂, —CO₂R, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NR₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), C₁-C₆ mercaptoalkyl, —CONH(C₁-C₆ alkyl), —CON(C₁-C₆ alkyl)₂, —CO₂H, —CO₂R, —OCOR, —OC(═O)OR, —OC(═O)NR, —OC(═S)OR, —OC(═S)NR, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; each of R₂, R₃ and R₄ independently represents hydrogen, or is selected from the group comprising halogen, —NO₂, —CN, —OH, —OR, —SR, —(C═O)NH₂, —CONR₂, —CO₂R, —SO₂R, oxo, carbonyl, amino, C₁-C₆ mercaptoalkyl, optionally substituted C₁-C₆ alkyl, optionally substituted C₁-C₆ heteroalkyl, imino, thioxo, carbonyl, aminoalkyl, fluoroalkyl, optionally substituted alkyl-aryl/heteroaryl, —O—C₁-C₆ haloalkyl, —O—C(R)₂—C₁-C₆ haloalkyl, —NR₂, -M-W, an optionally substituted C₃-C₈ saturated/unsaturated heterocyclyl, a cyclyl, optionally substituted C₃-C₈ saturated/unsaturated cycloalkyl, optionally substituted C₂-C₆ alkenyl, optionally substituted C₂-C₆ alkynyl, —C(═O)—R, —S—C₁-C₆ haloalkyl, —NH—C₁-C₆ haloalkyl, —NR₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, or any of R₂, R₃ and R₄ is absent (i.e. is H or is null, for example when referring to R₂ or R₄ bound to X, wherein L is absent and X is O, or S);
wherein at least one of R₂ and R₃ is not H or absent;
wherein M is selected from —O—, —S—, —NH—, —C(═O)—, —CONR—, —CNNR—, —CSNR—, —CONH—O—, —CONH—NH—, —NHCO—, —NHCSR—, —NHCN—, —NC(═O)O—, —NC(═O)N—, —NC(═S)O—, —NC(═S)N—, —SO₂—, —SO—, —SO₂O—, —SO₂NR—, —NHNR—, —CO₂—, —OCO—, —OC(═O)O—, —OC(═O)N—, —OC(═S)O— and —OC(═S)N—;
wherein W represents an optionally substituted C₃-C₈ cycloalkyl optionally substituted C₃-C₈ heterocyclyl optionally substituted aryl, optionally substituted alkyl-aryl/heteroaryl, or optionally substituted heteroaryl;
wherein each R independently represents hydrogen, or is selected from the group comprising optionally substituted C₁-C₁₀ alkyl, optionally substituted C1-C10 alkenyl/alkynyl, hydroxy, amino, —NH₂, —NR₂, —NH(C₁-C₆ alkyl), —N(C₁-C₆ alkyl)₂, C₁-C₆ alkoxy, —OR, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, optionally substituted C₃-C₁₀ cycloalkyl, optionally substituted C₁-C₁₀ alkaryl (e.g. benzyl), optionally substituted C₁-C₁₀ alkyl-C₃-C₁₀ cycloalkyl, optionally substituted C₃-C₁₀ heterocyclyl, optionally substituted heteroaryl, optionally substituted aryl, or a combination thereof; and wherein the compound comprises at least one R₁ representing the substituent described hereinabove (e.g. R₁ is not hydrogen); and wherein the compound encompasses any tautomer, any isotope (e.g. D-substituted compound via hydrogen-deuterium replacement) or a salt thereof. In some embodiments, the compound of the invention is represented by Formula IA or by Formula IB, as described herein, wherein at least one X₁ is N.

In one embodiment, provided herein is a composition comprising a compound of Formula I and a carrier. In one embodiment, provided herein a pharmaceutically acceptable salt of a compound of the invention such as but not limited to the compound of Formula I.

In some embodiments, the composition comprises the compound of the invention, a pharmaceutically acceptable salt thereof, tautomer or isotope thereof. In some embodiments, any compound of the invention such as but not limited to the compound of Formula I, a pharmaceutically acceptable salt, tautomer or isotope thereof is stable in an aqueous solution for at least one hour.

In some embodiments, any compound of the invention such as but not limited to the compound of Formula I, a pharmaceutically acceptable salt thereof or both is stable in an aqueous solution for at least 2 hours. In some embodiments, any compound of the invention such as but not limited to the compound of Formula I, a pharmaceutically acceptable salt thereof or both is stable in an aqueous solution for at least 5 hours. In some embodiments, any compound of the invention such as but not limited to the compound of Formula I, a pharmaceutically acceptable salt thereof or both is stable in an aqueous solution for at least 10 hours. In some embodiments, any compound of the invention such as but not limited to the compound of Formula I, a pharmaceutically acceptable salt thereof or both is stable in an aqueous solution for at least 24 hours. In some embodiments, any compound of the invention such as but not limited to the compound of Formula I, a pharmaceutically acceptable salt thereof or both is stable in an aqueous solution for at least 36 hours.

In another aspect, the present invention provides a compound represented by Formula IC:

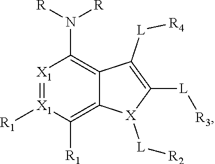

### or by Formula ID:

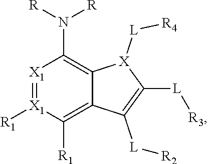

wherein R, R₁, R₂, R₃, R₄, X, X₁, and L, are as described herein. In one embodiment, provided herein is a composition comprising a compound represented by Formula IC and a carrier. In one embodiment, provided herein is a pharmaceutically acceptable salt of a compound represented by Formula ID.

In some embodiments, the compound of the invention is represented by Formula IE:

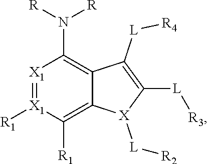

wherein R, R₁, R₂, R₃, R₄, X, X₁, and L, are as described herein.

In some embodiments, R₁ represents hydrogen, or a substituent comprising optionally substituted aryl, optionally substituted heteroaryl, optionally substituted heterocyclyl, and optionally substituted C₃-C₈ cycloalkyl or a combination thereof, wherein at least one R₁ within the compound of the invention represents the substituent, as described herein.

In some embodiments, R₁ represents a substituent selected from the group consisting of halogen, —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, and —OC(═S)NR, or a combination thereof.

In some embodiments, R₁ is selected from halo, nitro, cyano, —CONR₂, —(C═O)NH₂, —NRCOR, —NHCOH, haloalkyl, —SO₂R, —RSO₂, —CO₂R, —RCO₂R and —CF₃ or a combination thereof. In some embodiments, R₁ is selected from halo, nitro, cyano, —CON(C₁-C₆ alkyl)₂, —CONH (C₁-C₆ alkyl), —(C═O)NH₂, —N(C₁-C₆ alkyl)CO(C₁-C₆ alkyl), —NHCO(C₁-C₆ alkyl), —NHCOH, haloalkyl, —SO₂(C₁-C₆ alkyl), —(C₁-C₆ alkyl)SO₂, —CO₂(C₁-C₆ alkyl), —CO₂H, —(C₁-C₆ alkyl)CO₂(C₁-C₆ alkyl) and —CF₃ or a combination thereof.

In some embodiments, the compound of the invention is represented by any one of Formulae IA-IE, wherein R, R₂, R₃, R₄, X, X₁, and L, are as described herein, and R₁ is devoid of a cyclic group (e.g. aryl group). In some embodiments, R₁ is selected from halo, nitro, cyano, —CON(C₁-C₆ alkyl)₂, —CONH (C₁-C₆ alkyl), —(C═O)NH₂, —N(C₁-C₆ alkyl)CO(C₁-C₆ alkyl), —NHCO(C₁-C₆ alkyl), —NHCOH, haloalkyl, and —CF₃ or a combination thereof. In some embodiments, R₁ is selected from halo, cyano, —CON(C₁-C₆ alkyl)₂, —CONH (C₁-C₆ alkyl), and —(C═O)NH₂ or a combination thereof.

In some embodiments, the compound of the invention is represented by any one of Formulae IA-IE, wherein R, R₂, R₃, R₄, X₁, and R₁, are as described herein, X is N or NH, and L is a bond (e.g. is absent) or L comprises an optionally substituted C₁-C₆ alkyl, N, NH, S, and O, or any combination thereof. In some embodiments, L is absent. In some embodiments, L comprises C₁-C₅ alkyl, phosphonate, comprises phosphate, thiophosphate, —S(O)—, —S(O)₂—, —OS(O)₂—, —S(O)₂N—, or any combination thereof. In some embodiments, L comprises any of:

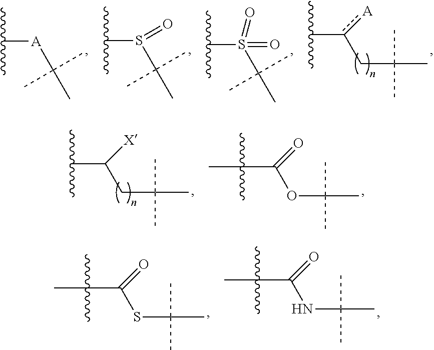

or a combination thereof, wherein a wavy bond represents an attachment point to the molecule, and wherein a dashed bond represents an attachment point to any of the substituents; wherein A comprises a heteroatom selected from N, O, S and P; or A is selected from H, —OR′, —SR′, —N(R′)₂, and NHR′; X′ comprises hydrogen, halogen, hydroxy, —CN, nitro, mercapto, amino, C₁-C₆ alkyl, C₁-C₆ haloalkyl, C₁-C₆ alkoxy, C₁-C₆ haloalkoxy, hydroxy(C₁-C₆ alkyl), amino(C₁-C₆ alkyl), optionally substituted C₃-C₈ cycloalkyl, —CO₂R, —CON(R′)o. 2, —CNNR′₂, —CSNR′₂, —CONH—OH, —CONH—NH₂, —NHCOR, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —CO₂H, or a combination thereof, wherein R′ is or comprises a C₁-C₆ alkyl. In some embodiments, L comprises —(C₁-C₆)₀₋₁-A-(C₁-C₆)₀₋₁—, wherein A comprises a heteroatom selected from N, O, S and P.

In some embodiments, L is absent or comprises any of a C₁-C₆ alkyl, —S(O)₂—, —C(O)—, —(C₁-C₆)—O—(C₁-C₆)₀₋₁—, —(C₁-C₆)—S—(C₁-C₆)₀₋₁—, —(C₁-C₆)—NH—(C₁-C₆)₀₋₁—, or any combination thereof.

In some embodiments, the compound of the invention is represented by any of Formulae IA-ID, wherein at least one X₁ is a heteroatom. In some embodiments, the compound of the invention is represented by any of Formulae IA-ID, wherein both X₁ are heteroatoms. In some embodiments, the compound of the invention is represented by any of Formulae IA-ID, wherein at least one X₁ comprises nitrogen. In some embodiments, the compound of the invention is represented by any of Formulae IA-ID, wherein both X₁ comprise nitrogen. As used herein, the term “heteroatom” is as described hereinbelow (e.g. being selected from N, NH, O, and S).

In some embodiments, the compound of the invention is represented by any of Formulae IA-ID, wherein both R bound to the exo-amine group are interconnected, so as to form a cyclic ring (e.g. 3-7, 4, 5, 6-membered aromatic, heteroaromatic ring and/or an aliphatic ring optionally comprising a heteroatom), a bicyclic ring and/or fused ring. In some embodiments, at least one R comprises an optionally substituted C₁-C₆ alkyl, wherein substituted is as described herein. In some embodiments, at least one R comprises —COR, wherein R is as described herein.

In some embodiments, the compound of the invention is represented by Formula IIA:

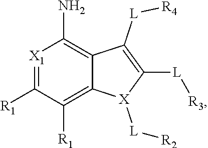

or by Formula IIB:

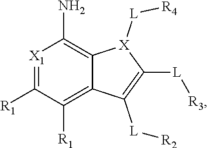

wherein R₂, R₃, R₄, X, X₁, L are as described herein; and at least one R₁ represents a substituent selected from the group consisting of halogen, —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, and —OC(═S)NR, or a combination thereof.

In some embodiments, the compound of the invention is represented by Formula IIC:

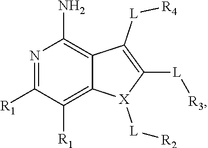

or by Formula IID:

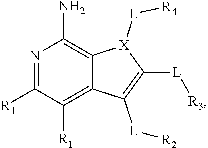

wherein R₂, R₃, R₄, L are as described herein; wherein X is N; and at least one R₁ represents a substituent selected from the group consisting of —CN, —(C═O)NH₂, —NHC(═O)H, —NHCOR, halogen, —NO₂, —OH, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, and —OC(═S)NR, or a combination thereof.

In some embodiments, (i) R₂ and R₃ or (ii) R₃ and R₄ are interconnected, so as to form a cyclic ring (e.g. 3-7, 4, 5, 6-membered aromatic, heteroaromatic ring and/or an aliphatic ring optionally comprising a heteroatom), a bicyclic ring and/or fused ring. In some embodiments, R₂ and R₁ are interconnected, so as to form an optionally substituted cyclic ring (e.g. 3-7, 4, 5, 6-membered aromatic, heteroaromatic ring and/or an aliphatic ring optionally comprising a heteroatom), a bicyclic ring and/or fused ring.

In some embodiments, the compound of the invention is represented by any of Formulae IIA-IID, wherein R₁, R₂, R₃, R₄, and L are as described herein, and wherein X is N.

In some embodiments, the compound of the invention is represented by Formula III.

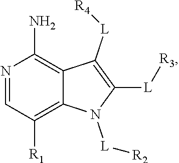

wherein R₁, R₂, R₃, R₄, and L are as described herein.

In some embodiments, the compound of the invention is represented by Formula III, wherein R₁ is selected from halo, nitro, cyano, —CON(C₁-C₆ alkyl)₂, —CONH (C₁-C₆ alkyl), —(C═O)NH₂, —N(C₁-C₆ alkyl)CO(C₁-C₆ alkyl), —NHCO(C₁-C₆ alkyl), —NHCOH, haloalkyl, and —CF₃ or a combination thereof. In some embodiments, R₁ is selected from halo, cyano, —CON(C₁-C₆ alkyl)₂, —CONH (C₁-C₆ alkyl), and —(C═O)NH₂ or a combination thereof.

In some embodiments, L is absent or comprises any of a —CH₂—, C₁-C₆ alkyl, —S(O)₂—, —C(O)—, or any combination thereof.

In some embodiments, the compound of the invention is represented by Formula IIIA:

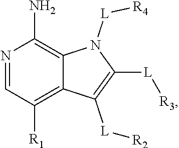

wherein R, R₁, R₂, R₃, R₄, and L are as described herein.

In some embodiments, the compound of the invention is represented by Formula IIIB:

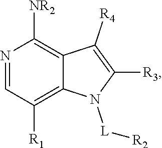

wherein R, R₁, R₂, R₃, R₄, and L are as described herein.

In some embodiments, the compound of the invention is represented by Formula IIIC:

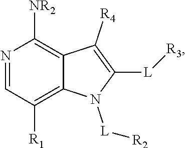

wherein R, R₁, R₂, R₃, R₄, and L are as described herein.

In some embodiments, the compound of the invention is represented by Formula IIID.

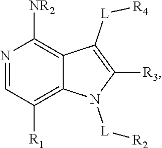

wherein R, R₁, R₂, R₃, R₄, and L are as described herein.

In some embodiments, the compound of the invention is represented by any one of Formulae IIIB-D, wherein R, R₁, R₂, R₃, R₄, are as described herein, and L comprises or is selected from the group consisting of a C₁-C₆ alkyl, C₁-C₂ alkyl, —S(O)₂—, —C(O)—, —(C₁-C₆)—O—(C₁-C₆)₀₋₁—, —(C₁-C₆)—S—(C₁-C₆)₀₋₁—, —(C₁-C₆)—NH—(C₁-C₆)₀₋₁—, or any combination thereof.

In some embodiments, the compound of the invention is represented by Formula III, wherein L is absent or comprises any of a C₁-C₂ alkyl, —S(O)₂—, —C(O)—, —(C₁-C₂)—O—(C₁-C₂)₀₋₁—, —(C₁-C₂)—S—(C₁-C₂)₀₋₁—, —(C₁-C₂)—NH—(C₁-C₂)₀₋₁—, or any combination thereof, wherein R₁ is selected from halo, cyano, —CON(C₁-C₆ alkyl)₂, —CONH (C₁-C₆ alkyl), HCONH—, and —(C═O)NH₂ or a combination thereof, wherein each of R₂ and R₃ independently represents hydrogen, or a substituent comprising optionally substituted heteroaryl, optionally substituted aryl, C₁-C₆ alkyl, C₁-C₆ haloalkyl, C₁-C₆ alkyl-NR₂, C₁-C₆ alkyl-OR, C₁-C₆ alkyl-SR, C₁-C₆ alkyl-Z, —Z, optionally substituted C₃-C₅ cycloalkyl, —(C₁-C₆)—O—(C₁-C₆)₀₋₁—, —(C₁-C₆)—S—(C₁-C₆)₀₋₁—, —(C₁-C₆)—NH—(C₁-C₆)₀₋₁—, optionally substituted bicyclic heteroaryl, optionally substituted bicyclic aryl, optionally substituted bicyclic heterocyclyl, optionally substituted bicyclic cycloalkyl, or a combination thereof, wherein Z is selected from the group consisting of —CO₂R, CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —CO₂R, CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, or a combination thereof, and wherein at least one of R₂ and R₃ represents the substituent (i.e. is not H); and wherein R₄ is as described herein.

In some embodiments, the compound of the invention is as described hereinabove, wherein R₂ and/or R₃ each independently comprises hydrogen, or represents a substituent comprising C₁-C₆ alkyl, C₁-C₆ haloalkyl, C₁-C₆ alkyl-NR₂, C₁-C₆ alkyl-OR, C₁-C₆ alkyl-SR, C₁-C₆ alkyl-Z, —Z, optionally substituted C₃-C₈ cycloalkyl, optionally substituted heteroaryl, optionally substituted aryl, optionally substituted bicyclic heteroaryl, optionally substituted bicyclic aryl, optionally substituted bicyclic heterocyclyl, optionally substituted bicyclic cycloalkyl, or a combination thereof; wherein substituted is by 1, 2, 3, 4, or 5 substituents selected from the group consisting of hydroxy(C₁-C₆ alkyl), halo, nitro, —NR₂, hydroxy, mercapto, C₁-C₆ haloalkyl, —SOR, —SO₂R, —SO₂N(R)₂, —SO₂OR, and —Z or a combination thereof. In some embodiments, Z is selected from the group consisting of —CO₂R, CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —CO₂R, CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, or a combination thereof.

In some embodiments, the compound of the invention is as described hereinabove, wherein R₂ and/or R₃ each independently comprises C₁-C₆ alkyl-Z, and/or —Z, wherein Z is selected from the group consisting of —CO₂R, CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —CO₂R, CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, or a combination thereof.

In some embodiments, R₂ represents a substituent comprising C₁-C₆ haloalkyl, C₁-C₆ alkyl-NR₂, —NR₂, C₁-C₆ alkyl-OR, C₁-C₆ alkyl-CONR₂, C₁-C₆ alkyl-COOR, C₁-C₆ alkyl-CNNR₂, C₁-C₆ alkyl-CSNR₂, (C₅-C₆) heteroaryl optionally substituted by one or more Y or a combination thereof. In some embodiments, Y is a substituent selected from C₁-C₆ haloalkyl, halo, fluoro, C₁-C₆ fluoroalkyl, (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkoxy, —NH₂, —NH(C₁-C₆ alkyl), —N(C₁-C₆ alkyl)₂, C₁-C₆ alkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, or a combination thereof. In some embodiments, Y represents between 1 and 2, between 2 and 3, between 3 and 4, between 4 and 5, substituents including any range or value therebetween. IN some embodiments Y is a single substituent.

In some embodiments, R₂ represents a substituent comprising C₁-C₆ haloalkyl, C₁-C₆ fluoroalkyl, C₁-C₆ alkyl-NR₂, C₁-C₆ alkyl-OR, C₁-C₆ alkyl-CONR₂, C₁-C₆ alkyl-COOR, C₁-C₆ alkyl-CNNR₂, C₁-C₆ alkyl-CSNR₂, C₅ or C₆ cycloalkyl optionally substituted by Y, C₅ or C₆ heteroaryl optionally substituted by Y, or a combination thereof. In some embodiments, R₂ represents a substituent comprising C₁-C₃ fluoroalkyl, C₁-C₂ fluoroalkyl, C₁-C₆ alkyl-NR₂, C₁-C₆ alkyl-OR, C₁-C₆ alkyl-CONR₂, C₁-C₆ alkyl-COOR, C₁-C₆ alkyl-CNNR₂, C₁-C₆ alkyl-CSNR₂ or a combination thereof.

In some embodiments, the compound of the invention is as described hereinabove, wherein R2 represents optionally substituted heteroaryl (e.g. comprising nitrogen, sulfur or both N and S as a heteroatom), or optionally substituted bicyclic heteroaryl, wherein substituted comprises one or more Y attached to any of the ring atoms. In some embodiments, the compound of the invention is as described hereinabove, wherein R₂ represents a C5-C6 heteroaryl, optionally comprising one or more Y.

In some embodiments, R₂ is represented by any one of:

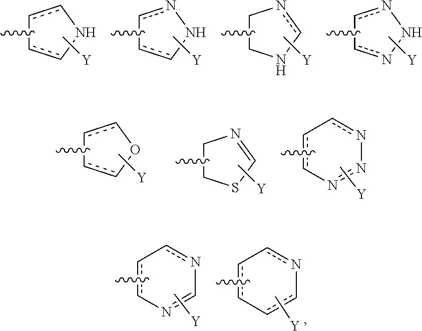

wherein Y is as described herein, and wherein R2 is attached to the scaffold (e.g. pyrrolopyridine-based scaffold) via any one of the ring atoms, as indicated by the wavy bond. In some embodiments, R₂ represents a heteroaromatic ring as described above, wherein the heteroaromatic ring is attached to the scaffold at ortho-position to heteroatom.

In some embodiments, R₂ is represented by any one of:

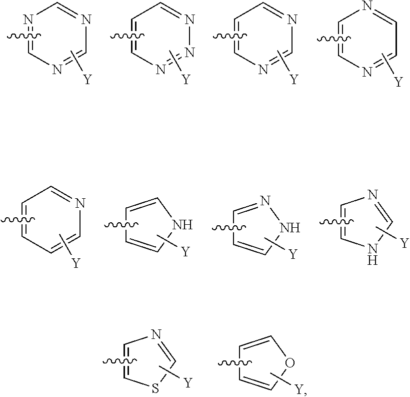

wherein Y is hydrogen or a substituent selected form the group consisting of halo (e.g. fluoro), C₁-C₆ haloalkyl, C₁-C₆ fluoroalkyl, —NO₂, —NH₂, —O(C₁-C₆ alkyl), —OMe, —NH(C₁-C₆ alkyl), —N(C₁-C₆ alkyl)₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, optionally substituted C₁-C₆ alkyl, —(C₁-C₆)OH, or a combination thereof.

In some embodiments, the compound of the invention is represented by any of Formulae I-III, wherein R₂ is represented by any one of

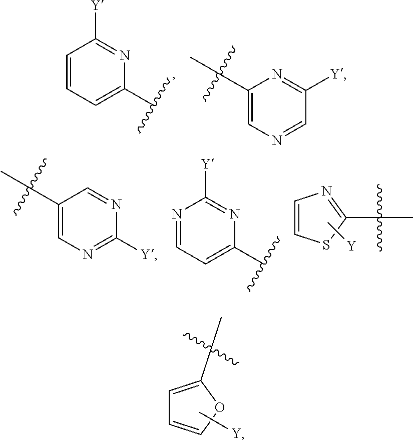

wherein Y is selected from the group consisting of halo (e.g. fluoro), and C₁-C₆ fluoroalkyl (e.g. C₁-C₃ fluoroalkyl or C₁-C₂ fluoroalkyl such as —CH₂F, —CHF₂, —CF₃), or a combination thereof, or Y is absent. In some embodiments, Y is F and/or —CHF₂. In some embodiments, Y is not an alkyl.

In some embodiments, R₂ is devoid of

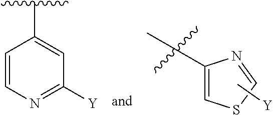

wherein Y is as described herein. In some embodiments, R₂ is devoid of 2-trifluoromethyl pyridine. In some embodiments, R₂ is devoid of an alkyl. In some embodiments, R₂ represents an aromatic and/or heteroaromatic substituent, wherein heteroaromatic is as described herein.

In some embodiments, the compound of the invention is represented by Formula III, wherein L is absent or comprises any of a C₁-C₆ alkyl, —S(O)₂—, —C(O)—, or any combination thereof; wherein R₁ is selected from halo, cyano, —CON(C₁-C₆ alkyl)₂, —CONH (C₁-C₆ alkyl), and —(C═O)NH₂ or a combination thereof, wherein R₃ represents hydrogen, or a substituent comprising unsaturated C₁-C₆ alkyl, optionally substituted alkaryl, optionally substituted heteroaryl, optionally substituted aryl, optionally substituted bicyclic heteroaryl, optionally substituted bicyclic heterocyclyl, optionally substituted bicyclic cycloalkyl, or a combination thereof; and wherein R₂ is represented by any one of:

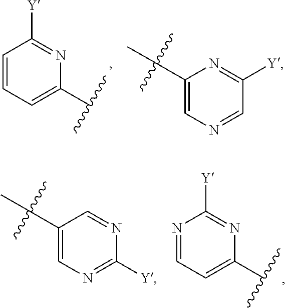

wherein Y is selected form the group consisting of halo (e.g. fluoro), —NO₂, —NH₂, —OMe, —(C═O)NH₂, —SO₂(C₁-C₆), and C₁-C₆ haloalkyl (e.g. —CH₂F, —CHF₂, —CF₃), or a combination thereof, or Y is absent; so that at least one of R₂ and R₃ represents the substituent; and wherein R₄ comprises hydrogen, halo, C₁-C₆ haloalkyl (e.g. —CF3, —CHF2, —CH2F), unsaturated C₁-C₆ alkyl, optionally substituted alkaryl, optionally substituted heteroaryl, optionally substituted aryl, optionally substituted bicyclic heteroaryl, optionally substituted bicyclic heterocyclyl, optionally substituted bicyclic cycloalkyl, or a combination thereof.

As used herein, the term “C₁-C₆ haloalkyl” refers to C₁-C₆ alkyl ad described herein substituted by 1, 2, 3, 4, 5, 6, 7, 8, 9, 10 halide atoms, wherein halide is selected from F, Br, C₁, and I, or a combination thereof. Non-limiting examples of C₁-C₆ haloalkyl include but are not limited to —CF3, —CHF2, —CH2F, —CH2-CF3, —CH2-CF3, —CH2—CH2F, —CC13, —CHBr2, —CHCl2, —CBr3, —CFBrCHFBr, —CH2I, —CH2Br, —CH2Cl, —CH2—CH2I, —CH2—CH2C₁, —CH2—CH2Br, or any combination thereof.

In some embodiments, the compound of the invention is represented by any of Formulae I-III, wherein R₃ represents hydrogen, or a substituent comprising unsaturated C₁-C₆ alkyl, optionally substituted alkaryl, optionally substituted heteroaryl, optionally substituted aryl, optionally substituted bicyclic heteroaryl, optionally substituted bicyclic heterocyclyl, optionally substituted bicyclic cycloalkyl, or a combination thereof.

In some embodiments, R₃ represents hydrogen, or a substituent comprising aryl optionally substituted by one or more Y; or C5 or C6 heteroaryl (e.g. pyrrole, furan, thiophene, thiazole, pyrazole, isothiazole, imidazole, pyridine, pyrimidine, etc.), or of a fused heterocyclic ring (e.g. indole, isoindole, benzofuran, benzothiophene, benzotriazole, quinoline, chromene, chroman, quinazoline), optionally substituted with one or more Y.

In some embodiments, R₃ comprises 7-10 bicyclic ring optionally substituted with one or more Y, wherein the bicyclic ring optionally comprises 1 or 2 heteroatoms (e.g. O, S or N or a combination thereof).

In some embodiments, R₃ comprises a fused 7-10 aliphatic ring optionally substituted with one or more Y. In some embodiments, fused 7-10 aliphatic ring comprises norbornane, bicyclooctane, bicyclodecane or a combination thereof. In some embodiments, at least one ring of the bicyclic ring comprises a heterocyclic aliphatic ring. In some embodiments, the heterocyclic aliphatic ring comprises pyrrolidine, oxirane, tetrahydrofuran, aziridine, pyran, dioxane, thiolane, oxathiolane, piperidine, and/or morpholine.

In some embodiments, R₃ comprises a fused 7-10 heterocyclic ring optionally substituted with one or more Y, wherein fused 7-10 heterocyclic ring is as described herein.

In some embodiments, R₃ comprises a phenyl ring or a pyridine ring optionally substituted by one or more Y. In some embodiments, R₃ comprises any of

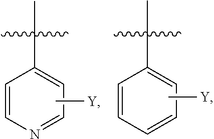

wherein Y is hydrogen or one or more substituent selected form the group consisting of optionally substituted C₁-C₆ alkyl, —NH₂, —O(C₁-C₆ alkyl), —OMe, —NH(C₁-C₆ alkyl), —N(C₁-C₆ alkyl)₂, —CN, —OH, —NHCOR, —NHNR₂, —NNR, C₁-C₆ haloalkyl, —(C₁-C₆)OH, or a combination thereof. In some embodiments, R₃ comprises pyridine, phenyl, para-methoxyphenyl, or is absent.

In some embodiments, the compound of the invention is represented by Formula III, wherein L is absent or comprises any of a C₁-C₆ alkyl, —S(O)₂—, —C(O)—, or any combination thereof; wherein R₁ is selected from halo, cyano, —CON(C₁-C₆ alkyl)₂, —CONH (C₁-C₆ alkyl), and —(C═O)NH₂ or a combination thereof, wherein R₃ represents hydrogen, or a substituent comprising a phenyl ring or a pyridine ring optionally substituted by one or more substituent selected from C₁-C₆ alkyl, —NH₂, —O(C₁-C₆ alkyl), —OMe, —NH(C₁-C₆ alkyl), —N(C₁-C₆ alkyl)₂, —CN, —OH, —NHCOR, —NHNR₂, —NNR, C₁-C₆ haloalkyl, —(C₁-C₆)OH, or a combination thereof, and wherein R₂ is represented by any one of:

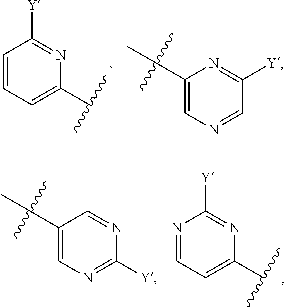

wherein Y is selected form the group consisting of halo (e.g. fluoro), —NO₂, —NH₂, —OMe, —(C═O)NH2, —SO₂(C₁-C₆), and C₁-C₆ haloalkyl (e.g. —CH₂F, —CHF₂, —CF₃), or a combination thereof, or Y is absent; and wherein R₄ comprises hydrogen, halo, unsaturated C₁-C₆ alkyl, C₁-C₆ haloalkyl (e.g. —CF3, —CHF2, —CH2F, —CH2-CF3, —CH2-CF3, —CH2—CH2F), optionally substituted alkaryl, optionally substituted heteroaryl, optionally substituted aryl, optionally substituted bicyclic heteroaryl, optionally substituted bicyclic heterocyclyl, optionally substituted bicyclic cycloalkyl, or a combination thereof.

In some embodiments, the compound of the invention is represented by Formula III, wherein L is absent or comprises any of a C₁-C₆ alkyl, —S(O)₂—, —C(O)—, or any combination thereof; wherein R₁ is selected from halo, cyano, —CON(C₁-C₆ alkyl)₂, —CONH (C₁-C₆ alkyl), and —(C═O)NH₂ or a combination thereof, wherein R₃ represents hydrogen, or a substituent comprising pyridine, phenyl, para-methoxyphenyl or a combination thereof; and wherein R₂ represents hydrogen or a substituent selected from any one of:

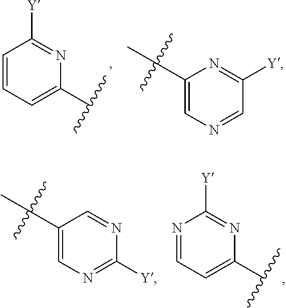

wherein Y is selected form the group consisting of halo (e.g. fluoro), —NO₂, —NH₂, —OMe, —(C═O)NH₂, —SO₂(C₁-C₆), and C₁-C₆ haloalkyl (e.g. —CH₂F, —CHF₂, —CF₃), or a combination thereof, or Y is absent; so that at least one of R₂ and R₃ represents the substituent; and wherein R₄ comprises hydrogen, halo, unsaturated C₁-C₆ alkyl, C₁-C₆ haloalkyl (e.g. —CF3, —CHF2, —CH2F), optionally substituted alkaryl, optionally substituted heteroaryl, optionally substituted aryl, optionally substituted bicyclic heteroaryl, optionally substituted bicyclic heterocyclyl, optionally substituted bicyclic cycloalkyl optionally substituted heterocyclyl or a combination thereof.

In some embodiments, the compound of the invention is represented by Formula IV:

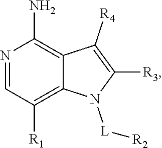

wherein R₁, R₂, R₃, R₄, and L are as described herein.

In some embodiments, the compound of the invention is represented by Formula IV, wherein L is absent or comprises any of a —CH₂—, C₁-C₆ alkyl, —S(O)₂—, —C(O)—, or any combination thereof; wherein R₁ is selected from halo, cyano, —CON(C₁-C₆ alkyl)₂, —CONH (C₁-C₆ alkyl), and —(C═O)NH₂ or a combination thereof, wherein R₃ represents hydrogen, or a substituent comprising pyridine, phenyl, para-methoxyphenyl or a combination thereof; and wherein R₂ is represented by any one of:

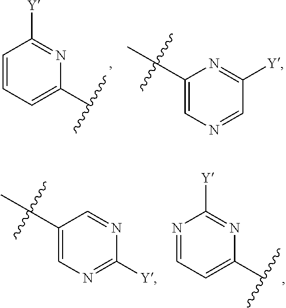

wherein Y′ is selected form the group consisting of halo (e.g. fluoro), —NO₂, —NH₂, —OMe, —(C═O)NH2, —SO₂(C₁-C₆), and C₁-C₆ haloalkyl (e.g. —CH₂F, —CHF₂, —CF₃), or a combination thereof, or Y′ is absent; and wherein R₄ comprises hydrogen, halo, unsaturated C₁-C₆ alkyl, C₁-C₆ haloalkyl (e.g. —CF3, —CHF2, —CH2F), optionally substituted alkaryl, optionally substituted heterocyclyl optionally substituted (C5-C6) heteroaryl, optionally substituted (C5-C6) heterocyclyl, optionally substituted C6 aryl, optionally substituted bicyclic heteroaryl, optionally substituted bicyclic heterocyclyl, optionally substituted bicyclic cycloalkyl, or a combination thereof.

In some embodiments, R₄ comprises hydrogen, halo,

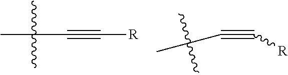

or a combination thereof. In some embodiments, R4 comprises C₁-C₆ haloalkyl (e.g. —CF3, —CHF2, —CH2F), C1-haloalkyl, C2-haloalkyl, C3-haloalkyl, C4-haloalkyl including any range therebetween. In some embodiments, R4 comprises an optionally substituted C₁-C₆ alkyl.

In some embodiments, R₄ comprises or is represented by

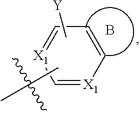

wherein X1 is selected from C, CH, and N; and wherein B comprises any one of:

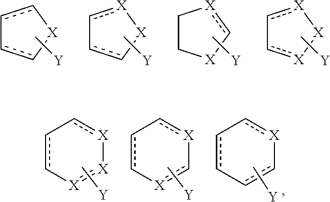

or wherein B is phenyl or C5-C6 cycloalkyl optionally substituted by one or more Y, or wherein B is absent; wherein each Y independently comprises a hydrogen accepting electron-withdrawing group (e.g. sulfone, methylsufone, amide, carboxy, ester); or an alkyl optionally substituted by hydroxy, amino, halo, mercapto or a combination thereof, or wherein Y is absent. In some embodiments, B is attached via one or two ring atoms to the C6 (hetero)aryl.

In some embodiments, Y is absent or is selected form the group consisting of (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, or a combination thereof, and wherein X is as described herein.

In some embodiments, R₄ comprises

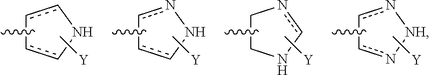

wherein Y is as described herein, and wherein any of the heterocyclic rings represented herein are variably attached to the scaffold (e.g. pyrrolopyridine-based scaffold), as indicated by the wavy bond.

In some embodiments, R₄ comprises (C₅-C₆)heterocyclyl, C₁-C₆ alkyl-(C₅-C₆)heterocyclyl or both. In some embodiments, R₄ comprises (C₅-C₆)heteroaryl (e.g. pyrazole, oxazole, imidazole) optionally substituted by one or more Y.

In some embodiments, the compound of the invention is represented by Formula V:

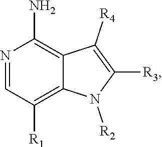

### or by Formula VA:

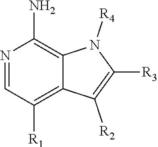

wherein:
R₁ is selected from halo (e.g. fluoro), cyano, and formamidyl; and R₂, and R₄ are as described herein; R₃ is H or is selected from halogen, —NO₂, —CN, —OH, —OR, —(C═O)NH₂, —CONR₂, —SR, —CO₂R, —SO₂R, optionally substituted C₁-C₆ alkyl, amino, oxo, imino, thioxo, carbonyl, aminoalkyl, —NH₂, —NH(C₁-C₆ alkyl), hydroxy(C₁-C₆ alkyl), C₁-C₆ haloalkyl, C₁-C₆ mercaptoalkyl, C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, and wherein the compound includes any salt thereof, any tautomer thereof; and any isotope thereof (e.g. hydrogen-deuterium replacement).

In some embodiments, the compound of the invention is represented by Formula V: wherein R₁ is selected from cyano and formamidyl (i.e., HCONH— or —(C═O)NH₂); wherein R₂ is H or is selected from C₁-C₆ haloalkyl (e.g. C1-C2 fluoroalkyl, such as —CF3, —CHF2, —CH2F), optionally substituted alkyl-aryl/heteroaryl, optionally substituted aromatic or heteroaromatic ring (e.g. a single 5-6 membered ring or a bicyclic ring);
wherein R₃ is H, or (i) if R₂ is an optionally substituted alkyl-aryl/heteroaryl or an optionally substituted aromatic or heteroaromatic ring, then R₃ is selected from halogen, —NO₂, —CN, —OH, —(C═O)NH₂, —CONR₂, —SR, —CO₂R, —SO₂R, optionally substituted C₁-C₆ alkyl, amino, oxo, carbonyl, aminoalkyl, —NR₂, —NH(C₁-C₆ alkyl), hydroxy(C₁-C₆ alkyl), C₁-C₆ haloalkyl, C₁-C₆ mercaptoalkyl, C₁-C₆ haloalkyl and C₁-C₆ heteroalkyl; or (ii) if R₂ is C₁-C₆ haloalkyl, then R₃ is selected from optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof;
and wherein R₄ is H or is selected from halogen (e.g. F or Br); —OR (e.g. —OMe, —OEt, —OPr); —O—C₁-C₆ haloalkyl; —O—C(R)₂—C₁-C₆ haloalkyl

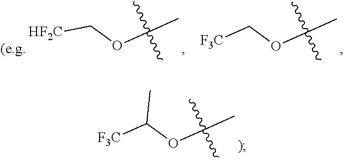

C₁-C₆ haloalkyl

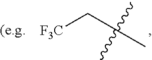 [SMILES: *CC(F)(F)F]

optionally deuterated); optionally substituted C₁-C₆ alkyl (e.g. ethyl, deuterated ethyl,

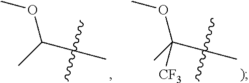

-M-W, wherein M is selected from —O—, —S—, —NH—, —C(═O)—, —CONR—, —CNNR—, —CSNR—, —CONH—O—, —CONH—NH—, —NHCO—, —NHCSR—, —NHCN—, —NC(═O)O—, —NC(═O)N—, —NC(═S)O—, —NC(═S)N—, —SO₂—, —SO—, —SO₂O—, —SO₂NR—, —NHNR—, —CO₂—, —OCO—, —OC(═O)O—, —OC(═O)N—, —OC(═S)O— and —OC(═S)N—, and wherein W represents an optionally substituted C₃-C₈ cycloalkyl

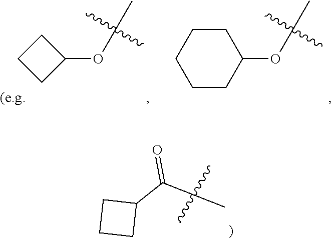 [SMILES: *OC1CCC1 ; *OC1CCCCC1 ; *C(=O)C1CCC1]

optionally substituted C₃-C₈ heterocyclyl

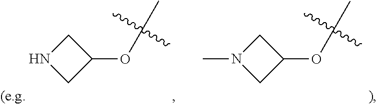

optionally substituted aryl, optionally substituted alkyl-aryl/heteroaryl, or optionally substituted heteroaryl; an optionally substituted C₃-C₈ saturated/unsaturated heterocyclyl

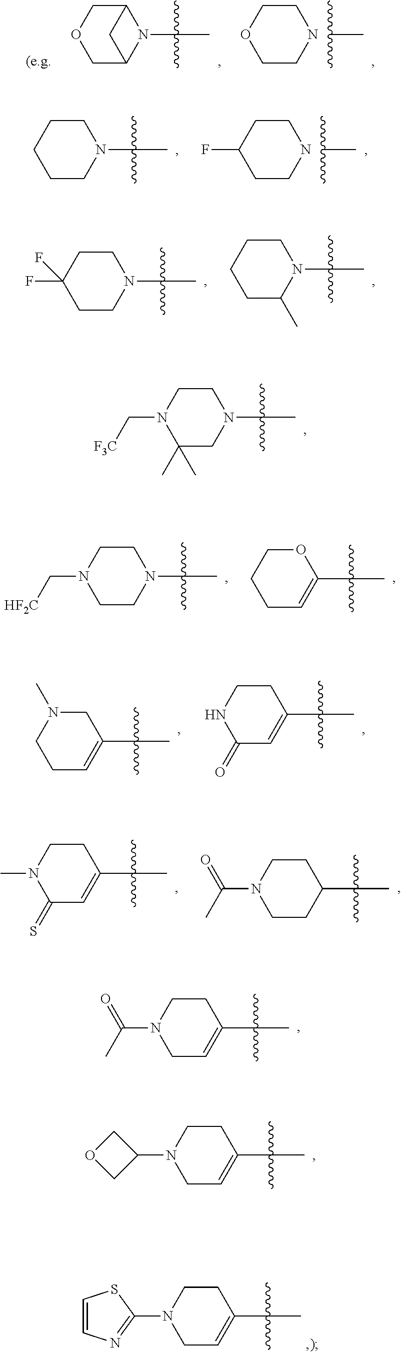

optionally substituted C₃-C₈ saturated/unsaturated cycloalkyl

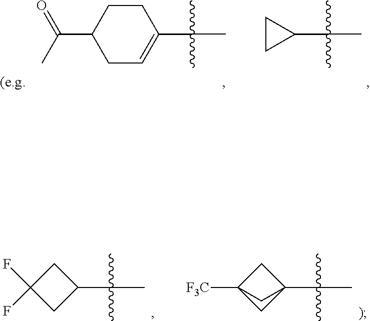

optionally substituted C₂-C₆ alkenyl

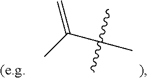 [SMILES: *C(=C)C]

—CN, optionally substituted C₂-C₆ alkynyl (e.g. acetylene,

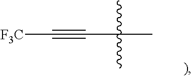

### —C(═O)—R

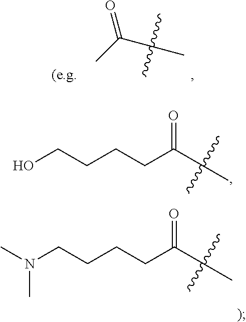

—SR; —S—C₁-C₆ haloalkyl; —NH—C₁-C₆ haloalkyl; —NO₂, —OH, —(C═O)NH₂, —CONR₂, —CO₂R, —SO₂R, —NH₂, —NH(C₁-C₆ alkyl), hydroxy(C₁-C₆ alkyl), optionally substituted heteroaryl (e.g. pyridine/pyrazole/imidazole/thiazole/furan/pyrrole/thiophene optionally substituted by one or more Y,

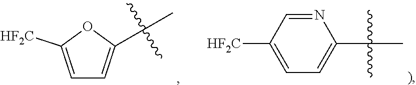

optionally substituted aryl (e.g., phenyl, phenyl substituted by one or more Y,

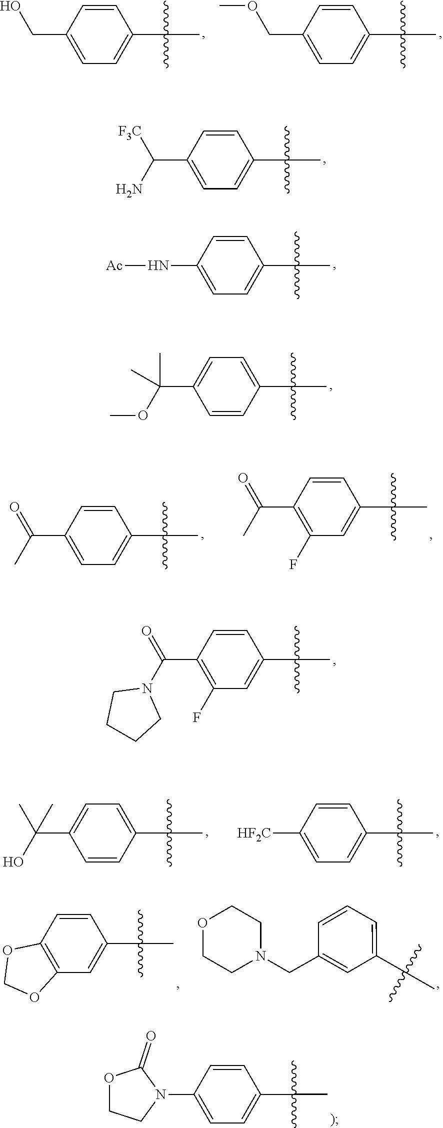

optionally substituted heterocyclyl; optionally substituted bicyclic heteroaryl

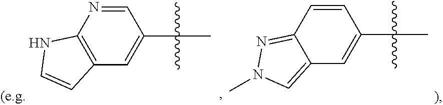

optionally substituted bicyclic aryl

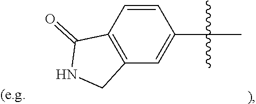

optionally substituted bicyclic heterocyclyl, optionally substituted bicyclic cycloalkyl, or a combination thereof; and if R₃ is H then R₂ is not H; wherein Y is selected from haloalkyl, C₁-C₆ haloalkyl, alkoxy, —C(═O)R, heteroalkyl, aminoalkyl, (C₀-C₆)alkyl-aryl, (C₀-C₆)alkyl-heteroaryl, (C₀-C₆)alkyl-(C₃-C₈) cycloalkyl, O—C₁-C₆ haloalkyl, —O—C(R)₂—C₁-C₆ haloalkyl and optionally substituted C₁-C₆ alkyl; -M-W, optionally substituted C₃-C₈ heterocyclyl, amino, oxo, imino, thioxo, carbonyl, halogen, —NO₂, —CN, —OR, —(C═O)NH₂, —CONR₂, —CNNR₂, —CSNR₂, —CONH—OR, RCONH—, —NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, optionally substituted C₁-C₆ alkyl, —NR₂, —NH(C₁-C₆ alkyl), —N(C₁-C₆ alkyl)₂, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆alkyl-SR, —CONH(C₁-C₆ alkyl), —CON(C₁-C₆ alkyl)₂, —CO₂H, —CO₂R, RCO₂—, —OCOR, —OCOR, —OC(═O)OR, —OC(═O)NR, —OC(═S)OR, —OC(═S)NR, or a combination thereof, wherein any one of haloalkyl, alkoxy, heteroalkyl, aminoalkyl, (C₀-C₆)alkyl-aryl, (C₀-C₆)alkyl-heteroaryl and (C₀-C₆)alkyl-(C₃-C₈) cycloalkyl is optionally substituted.

In some embodiments, the compound of the invention is represented by Formula V, wherein R₃ is H, R₁ is cyano or formamidyl; R₂ is selected from C₁-C₆ fluoroalkyl (e.g. C₁-C₃ fluoroalkyl, such as —CF3, —CHF2, —CH2F), heteroaromatic ring (e.g. a single ring or a fused ring) optionally substituted by one or more substituents, optionally wherein the substituents are interconnected so as to form a 5-, or 6-membered aliphatic, unsaturated or aromatic ring, and R₄ is H or is selected from halogen (e.g. F or Br); —OR (e.g. —OMe, —OEt, —OPr); —O—C₁-C₆ haloalkyl; —O—C(R)₂—C₁-C₆ haloalkyl

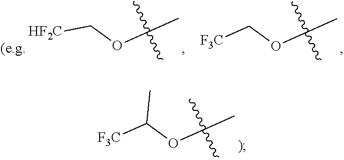

C₁-C₆ haloalkyl

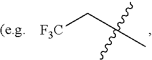 [SMILES: *CC(F)(F)F]

optionally deuterated); optionally substituted C₁-C₆ alkyl (e.g. ethyl, deuterated ethyl,

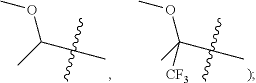

-M-W, wherein M is selected from —O—, —S—, —NH—, —C(═O)—, —CONR—, —CNNR—, —CSNR—, —CONH—O—, —CONH—NH—, —NHCO—, —NHCSR—, —NHCN—, —NC(═O)O—, —NC(═O)N—, —NC(═S)O—, —NC(═S)N—, —SO₂—, —SO—, —SO₂O—, —SO₂NR—, —NHNR—, —CO₂—, —OCO—, —OC(═O)O—, —OC(═O)N—, —OC(═S)O— and —OC(═S)N—, and wherein W represents an optionally substituted C₃-C₈ cycloalkyl

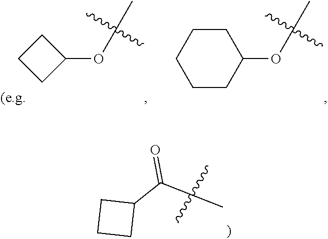 [SMILES: *OC1CCC1 ; *OC1CCCCC1 ; *C(=O)C1CCC1]

optionally substituted C₃-C₈ heterocyclyl

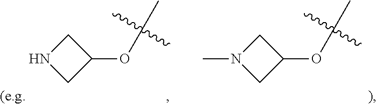

optionally substituted aryl, optionally substituted alkyl-aryl/heteroaryl, or optionally substituted heteroaryl; an optionally substituted C₃-C₈ saturated/unsaturated heterocyclyl

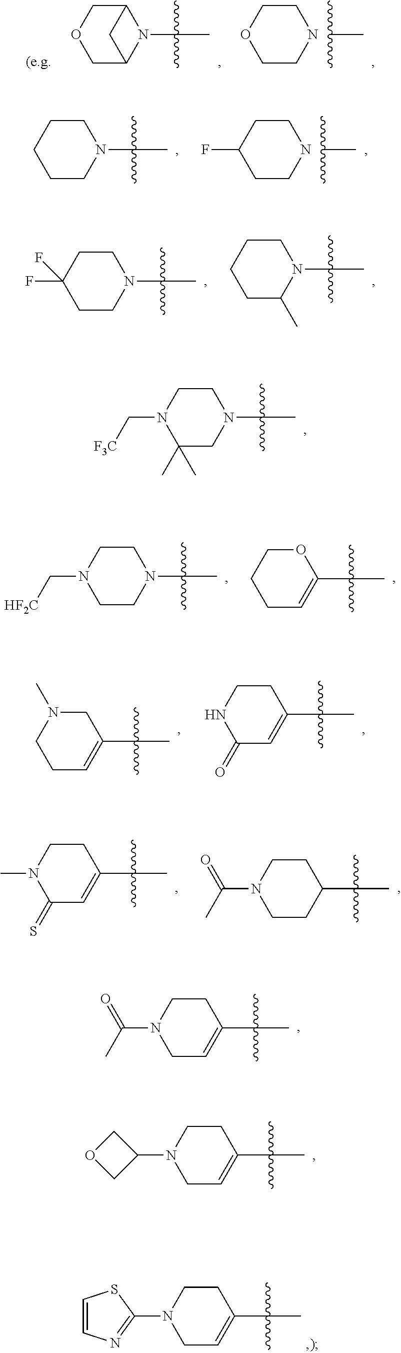

optionally substituted C₃-C₈ saturated/unsaturated cycloalkyl

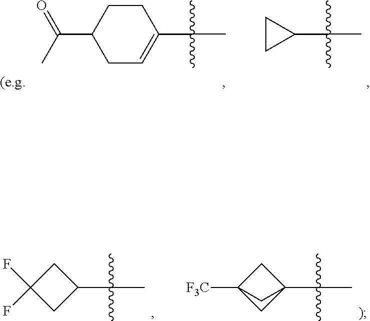

optionally substituted C₂-C₆ alkenyl

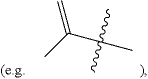 [SMILES: *C(=C)C]

—CN, optionally substituted C₂-C₆ alkynyl (e.g. acetylene,

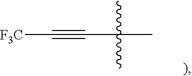

### —C(═O)—R

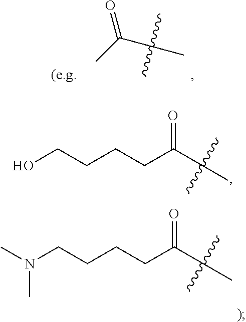

—SR; —S—C₁-C₆ haloalkyl; —NH—C₁-C₆ haloalkyl; —NO₂; —OH; —CONR₂; —CONR₂; —CO₂R; —SO₂R; —NH₂; —NR₂; —NH(C₁-C₆ alkyl); hydroxy(C₁-C₆ alkyl); optionally substituted heteroaryl (e.g. pyridine/pyrazole/imidazole/thiazole/furan/pyrrole/thiophene optionally substituted by one or more Y,

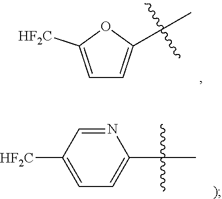

optionally substituted aryl (e.g., phenyl, phenyl substituted by one or more Y,

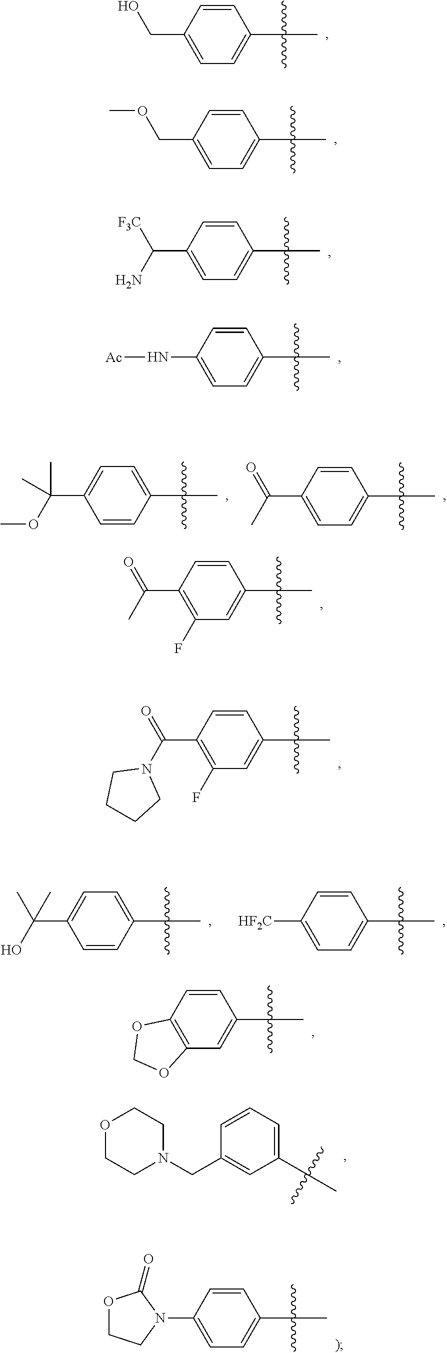

optionally substituted heterocyclyl; optionally substituted bicyclic heteroaryl

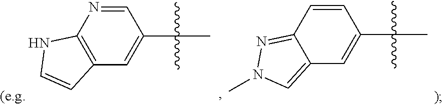

optionally substituted bicyclic aryl

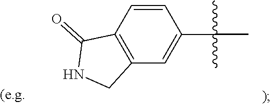

optionally substituted bicyclic heterocyclyl; optionally substituted bicyclic cycloalkyl, or a combination thereof.

In some embodiments, the compound of the invention is represented by Formula V, wherein R₃ is H, R₁ is cyano or formamidyl; R₂ is a C1-C3 fluoroalkyl, (such as —CH2F, —CF3), or a 5-6 membered heteroaromatic ring (e.g. pyridine, pyrimidine, thiazole, furane, pyrazole) substituted by one or more substituents, wherein at least one of the one or more substituents is selected from —F, amino (e.g. —NH₂), fluoroalkyl (e.g. C₁-C₃ fluoroalkyl, such as —CH2F optionally deuterated fluoroalkyl), and fluoroalkyl substituted by one or more substituents which is not fluoro; and R4 is as described above for Formula V. In some embodiments, the compound of the invention is represented by Formula V, wherein R₃ is H, R₁ is cyano or formamidyl; R₂ is a C1-C3 fluoroalkyl (such as —CH2F), or a 5-6 membered heteroaromatic ring substituted by —F or C1-C3 fluoroalkyl; and R4 is selected from haloalkyl (e.g. fluoroalkyl), halo, —OR, —C(═O)—R, an optionally substituted C₃-C₈ heterocyclyl, an optionally substituted C₃-C₈ heterocyclyl comprising an unsaturated bond, and an optionally substituted aryl.

In some embodiments, the compound of the invention is represented by Formula V, wherein R₃ is H, R₁ is cyano or formamidyl; R₂ is a C1-C3 fluoroalkyl (such as —CH2F), or a 5-6 membered heteroaromatic ring substituted by —F or C1-C3 fluoroalkyl; and R4 is selected from haloalkyl (e.g. fluoroalkyl), halo, —OR (e.g. methoxy), —C(═O)—R

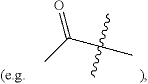 [SMILES: *C(C)=O]

substituted C₃-C₆ heterocyclyl

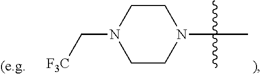 [SMILES: *N1CCN(CC(F)(F)F)CC1]

substituted C₃-C₆ cycloalkyl

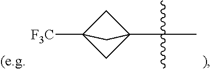 [SMILES: *C12CC(C(F)(F)F)(C1)C2]

substituted C₃-C₆ heterocyclyl comprising an unsaturated bond

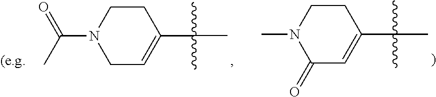

and a substituted aryl

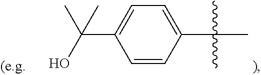 [SMILES: *c1ccc(C(C)(C)O)cc1]

and wherein any of the substituted C₃-C₈ heterocyclyl and aryl is substituted by at least one substituent selected from haloalkyl, C₁-C₆ haloalkyl, alkoxy, C₁-C₆ hydroxyalkyl, oxo, optionally substituted C₁-C₆ alkyl and —C(═O)—R.

In some embodiments, the compound of the invention is represented by Formula VB:

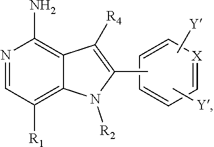

wherein X is N or CH; wherein each Y′ is independently hydrogen or one or more substituent selected from the group consisting of optionally substituted C₁-C₆ alkyl, —OR, —NR₂, —O(C₁-C₆ alkyl), —OMe, —NH(C₁-C₆ alkyl), —N(C₁-C₆ alkyl)₂, —CN, —OH, —NHCOR, —NHNR₂, —NNR, C₁-C₆ haloalkyl, —(C₁-C₆)OH, or wherein both Y′ are interconnected so as to form a ring (e.g. a 5-6 membered ring, such as

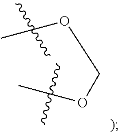

wherein R₁ is selected from halo, cyano, - and formamidyl-(C═O)NH₂; wherein R₂ is selected from H and C₁-C₆ fluoroalkyl (e.g. C₁-C₂ fluoroalkyl, such as —CF3, —CHF2, —CH2F); and wherein R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

In some embodiments, the compound of the invention is represented by Formula VB:

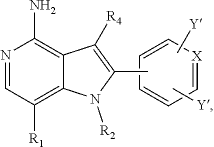

wherein X is N or CH; wherein each Y′ is independently hydrogen or one or more substituent selected from the group consisting of optionally substituted C₁-C₆ alkyl, —OR, —NR₂, —O(C₁-C₆ alkyl), —OMe, —NH(C₁-C₆ alkyl), —N(C₁-C₆ alkyl)₂, —CN, —OH, —NHCOR, —NHNR₂, —NNR, C₁-C₆ haloalkyl, —(C₁-C₆)OH, or wherein both Y′ are interconnected so as to form a ring (e.g. a 5-6 membered ring, such as

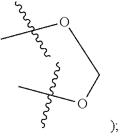

wherein R₁ is selected from halo, cyano, - and formamidyl-(C═O)NH₂; wherein R₂ is selected from H and C₁-C₆ fluoroalkyl (e.g. C₁-C₂ fluoroalkyl, such as —CF3, —CHF2, —CH2F); and wherein R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl) and hydroxy(C₁-C₆ alkyl).

In some embodiments, the compound of the invention is represented by Formula VA, wherein R₃ is H, R₁ is cyano or formamidyl; R₂ is a C₁-C₃ fluoroalkyl, (such as —CH2F, —CF3), or a 5-6 membered heteroaromatic ring (e.g. pyridine, pyrimidine, thiazole, furane, pyrazole) substituted by one or more substituents, wherein at least one of the one or more substituents is selected from —F, amino (e.g. —NH₂), fluoroalkyl (e.g. C₁-C₃ fluoroalkyl, such as —CH2F optionally deuterated fluoroalkyl), and fluoroalkyl substituted by one or more substituents which is not fluoro; and wherein R₄ is selected from H, halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

In some embodiments, the compound of the invention is represented by Formula V, wherein R₃ is H, R₁ is cyano or formamidyl; R₂ is a C1-C3 fluoroalkyl, (such as —CH2F, —CF3), or a 5-6 membered heteroaromatic ring (e.g. pyridine, pyrimidine, thiazole, furane, pyrazole) substituted by one or more substituents, wherein at least one of the one or more substituents is selected from —F, amino (e.g. —NH₂), fluoroalkyl (e.g. C1-C3 fluoroalkyl, such as —CH2F optionally deuterated fluoroalkyl), and fluoroalkyl substituted by one or more substituents which is not fluoro; and wherein R₄ is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl; and if R2 is the 5-6 membered heteroaromatic ring, then R4 is selected from H, halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); wherein W represents an optionally substituted C₃-C₈ cycloalkyl, or optionally substituted C₃-C₈ heterocyclyl.

In some embodiments, the compound of the invention is represented by any of Formulae V-VB, wherein R₃ is hydrogen; and wherein R₂ is selected from pyridine, pyrimidine and a 5 membered heteroaromatic ring (e.g. thiazole, furane or pyrazole) substituted by one or two Y″, wherein each Y″ is independently selected from fluoro, —(C═O)NH₂ and C₁-C₆ fluoroalkyl (e.g. —CH₂F, —CHF₂, —CF₃); and wherein R₄ is as described hereinabove for Formula V.

In some embodiments, the compound of the invention is represented by any of Formulae V-VB, wherein R₄ comprises

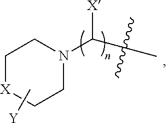

wherein Y, X and X′ are as described hereinabove and wherein n is 0, 1 or 2.

In some embodiments, the compound of the invention is represented by any of Formulae V-VB, wherein R₄ comprises

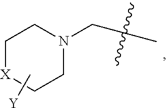

wherein Y is selected from —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, —CO₂R, —OCOR, —OCOR, —OC(═O)OR, —OC(═O)NR, —OC(═S)OR, —OC(═S)NR, or a combination thereof or wherein Y is absent, and wherein R is as described herein. In some embodiments, Y is attached to a heteroatom.

In some embodiments, the compound of the invention is represented by any of Formulae V-VB, wherein R₄ comprises

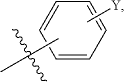

wherein Y is absent or is selected form the group consisting of (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, halogen, —NO₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, —CONH(C₁-C₆ alkyl), —CON(C₁-C₆ alkyl)₂, —CO₂H, —CO₂R, —OCOR, —OCOR, —OC(═O)OR, —OC(═O)NR, —OC(═S)OR, —OC(═S)NR, or a combination thereof.

In some embodiments, the compound of the invention is represented by any of Formulae V-VB, wherein R₄ comprises

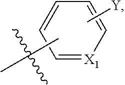

wherein X1 is CH or N; and wherein Y is selected from hydrogen, —NHCOR, —SO₂R, C₁-C₆ alkyl-NR₂, C₁-C₆ alkyl-OR, —CO₂R, C₁-C₆ alkyl-NR₂ substituted by one or more X′, C₁-C₆ alkyl-OR substituted by one or more X′, C₁-C₆ alkyl —SR substituted by one or more X′, (C₀-C₆)alkyl-aryl, (C₀-C₆)alkyl-heteroaryl, (C₀-C₆)alkyl-(C₃-C₈) cycloalkyl, optionally substituted C₃-C₈ heterocyclyl, optionally substituted C₁-C₆ alkyl, —NO₂, —CN, halo, —CF3, —OH, —(C═O)NH₂, —CONR₂, —CNNR₂, —CSNR₂, —CONH—OH, —CONH—NH₂, —NHCSR, —NHCNR, —NC(═O)OR, —NC(═O)NR, —NC(═S)OR, —NC(═S)NR, —SOR, —SR, —SO₂OR, —SO₂N(R)₂, —NHNR₂, —NNR, C₁-C₆ haloalkyl, —OCOR, —OCOR, —OC(═O)OR, —OC(═O)NR, —OC(═S)OR, —OC(═S)NR, C₁-C₆ haloalkyl, —NH₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-SR or a combination thereof. In some embodiments, Y comprises a hydrogen accepting electron-withdrawing group (e.g. sulfone, methylsufone, amide, carboxy, ester); or an alkyl optionally substituted by hydroxy, amino, halo, mercapto or a combination thereof, or wherein Y is absent.

In some embodiments, the compound of the invention is represented by any of Formulae V-VB, wherein R₄ is as described hereinabove (e.g. an aryl, a heteroaryl, a fused aryl, or a fused heteroaryl), substituted by at least one Y, wherein Y comprises:

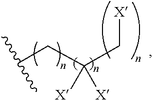

wherein each n independently represents an integer being between 0 and 10, between 0 and 1, between 1 and 2, between 2 and 3, between 3 and 5, between 5 and 10, including any range therebetween; and wherein each X′ independently comprises or is selected from hydrogen, halogen, hydroxy, —CN, nitro, mercapto, amino, C₁-C₆ alkyl, C₁-C₆ haloalkyl, C₁-C₆ alkoxy, C₁-C₆ haloalkoxy, hydroxy(C₁-C₆ alkyl), amino(C₁-C₆ alkyl), optionally substituted C₃-C₈ cycloalkyl, —CO₂R, —CON(R)₀₋₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —CO₂H, or a combination thereof, wherein R is as described hereinabove.

In some embodiments, the compound of the invention is represented by any of Formulae V-VB, wherein R₄ comprises

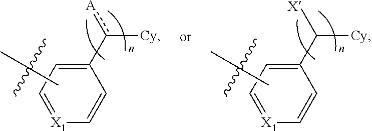

wherein A comprises a heteroatom selected from N, O, S and P; X′ comprises hydrogen, halogen, hydroxy, —CN, nitro, mercapto, amino, C₁-C₆ alkyl, C₁-C₆ haloalkyl, C₁-C₆ alkoxy, C₁-C₆ haloalkoxy, hydroxy(C₁-C₆ alkyl), amino(C₁-C₆ alkyl), optionally substituted C₃-C₈ cycloalkyl, —CO₂R, —CON(R′)₀₋₂, —CNNR′₂, —CSNR′₂, —CONH—OH, —CONH—NH₂, —NHCOR, —NHCSR′, —NHCNR′, —NC(═O)OR′, —NC(═O)NR′, —NC(═S)OR′, —NC(═S)NR′, —SO₂R′, —SOR′, —SR′, —SO₂OR′, —SO₂N(R′)₂, —CO₂H, or a combination thereof, wherein R′ is C₁-C₆ alkyl; and wherein Cy comprises optionally substituted (C₅-C₆)heterocyclyl, optionally substituted C₁-C₆ alkyl-(C₅-C₆)heterocyclyl, optionally substituted alkaryl, optionally substituted (C₅-C₆)heteroaryl, optionally substituted aryl, optionally substituted bicyclic heteroaryl, optionally substituted bicyclic heterocyclyl, optionally substituted cycloalkyl, optionally substituted bicyclic cycloalkyl or a combination thereof; and wherein n is 0, 1, 2, 3, 4, or 5.

In some embodiments, the compound of the invention is represented by any of Formulae V-VB, wherein R₄ comprises

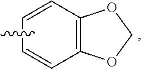

optionally substituted by any of Y,

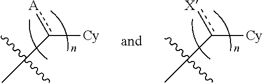

or a combination thereof.

In some embodiments, the compound of the invention is represented by any of Formulae V-VB, wherein R₄ comprises any of pyridine, phenyl, pyrimidine, pyridazine, pyrazine, triazine, pyrazole, pyrrole, triazole, imidazole, indole, iso-indole, indazole, benzimidazole, azaindazole, purine, azaindole, and indoline, optionally substituted by any of Y,

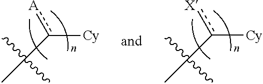

and or a combination thereof.

In some embodiments, the compound of the invention is represented by any of Formulae V-VB, wherein R₄ comprises any of isoindolin-1-one, isoindolin-2-one, optionally substituted by any of Y,

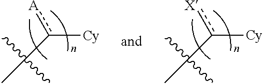

or a combination thereof.

In some embodiments, the compound of the invention is represented by any of Formulae V-VB, wherein R₄ comprises any of:

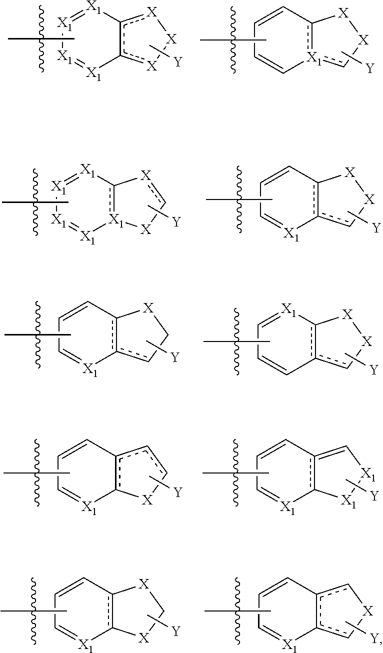

wherein X1 is CH or N; and wherein X and Y are as described herein.

In some embodiments, the compound of the invention is represented by any of Formulae V-VB, wherein R₄ comprises any of

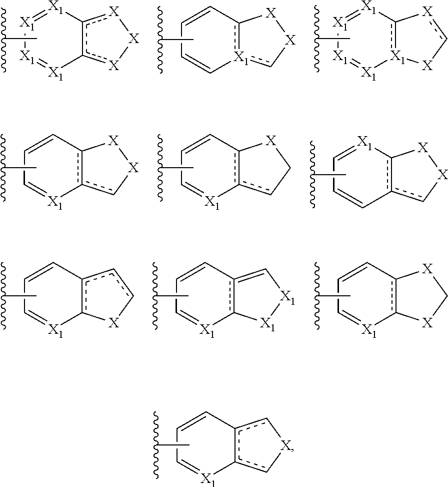

optionally substituted by any of

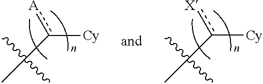

or a combination thereof, wherein X1 is CH or N; and wherein X is as described herein.

In some embodiments, the compound of the invention is as described hereinabove, wherein at least R1 and an additional substituent (e.g. any ofR², R3, and R4) are present within the molecule.

In some embodiments, the compound of the invention is represented by Formula V, wherein R1 and at least one of R2, R3, and R4 are substituents. In some embodiments, the compound of the invention is represented by Formula V, wherein R1 and at least two of R2, R3, and R4 are substituents. In some embodiments, the compound of the invention is represented by Formula V, wherein R1, R2, and R4 are substituents, and wherein R3 is H. In some embodiments, the compound of the invention is represented by Formula V, wherein R1, R2, and R3 are substituents, and wherein R4 is H. In some embodiments, the compound of the invention is represented by Formula V, wherein R1, R4, and R3 are substituents, and wherein R2 is H. In some embodiments, the compound of the invention is represented by Formula V, wherein R1, and R4 are substituents, and wherein R2 and R3 are H. In some embodiments, the compound of the invention is represented by Formula V, wherein R3 or R4 are H.

In some embodiments, the compound of the invention is represented by Formula VI:

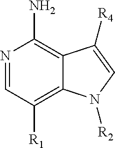

wherein R₁ is selected from cyano and formamidyl; wherein R₂ is H or is selected from C₁-C₆ haloalkyl (e.g. C1-C2 fluoroalkyl, such as —CF3, —CHF2, —CH2F), optionally substituted alkyl-aryl/heteroaryl, optionally substituted aromatic or heteroaromatic ring (e.g. a single 5-6 membered ring or a bicyclic ring);
and wherein R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

In some embodiments, the compound of the invention is represented by Formula VI, wherein R₁ is cyano or formamidyl; R₂ is a C1-C3 fluoroalkyl, (such as —CH2F, —CF3), or a 5-6 membered heteroaromatic ring (e.g. pyridine, thiazole, furane) substituted by one or more substituents, wherein at least one of the one or more substituents is selected from —F, amino (e.g. —NH₂), fluoroalkyl (e.g. C1-C3 fluoroalkyl, such as —CH2F optionally deuterated fluoroalkyl), and fluoroalkyl substituted by one or more substituents which is not fluoro; and R4 is as described above for Formula VI.

In some embodiments, the compound of the invention is represented by Formula VI, wherein R₁ is cyano or formamidyl; R₂ is a C1-C3 fluoroalkyl (such as —CH2F), or a 5-6 membered heteroaromatic ring substituted by —F or C1-C3 fluoroalkyl; and R4 is selected from haloalkyl (e.g. fluoroalkyl), halo, —OR (e.g. methoxy), —C(═O)—R

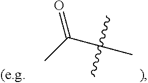 [SMILES: *C(C)=O]

substituted C₃-C₆ heterocyclyl

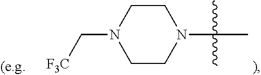 [SMILES: *N1CCN(CC(F)(F)F)CC1]

substituted C₃-C₆ cycloalkyl

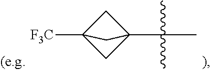 [SMILES: *C12CC(C(F)(F)F)(C1)C2]

substituted C₃-C₆ heterocyclyl comprising an unsaturated bond

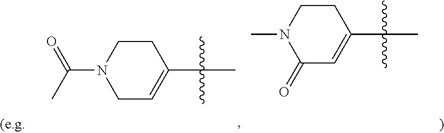

and a substituted aryl

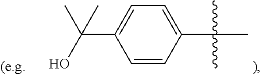 [SMILES: *c1ccc(C(C)(C)O)cc1]

and wherein any of the substituted C₃-C₈ heterocyclyl and aryl is substituted by at least one substituent selected from haloalkyl, C₁-C₆ haloalkyl, alkoxy, C₁-C₆ hydroxyalkyl, oxo, optionally substituted C₁-C₆ alkyl and —C(═O)—R.

In some embodiments, the compound of the invention is represented by Formula VI, wherein R₁ is cyano or formamidyl; R₂ is a C1-C3 fluoroalkyl (such as —CH2F), or a 5-6 membered heteroaromatic ring substituted by —F or C1-C3 fluoroalkyl; R4 is as described for Formula V; and if R2 is the 5-6 membered heteroaromatic ring, then R4 is selected from H, halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH2; —CONR₂; —CO₂R; —SO₂R; —NH₂; —NR₂; —NH(C₁-C₆ alkyl); hydroxy(C₁-C₆ alkyl); wherein W represents an optionally substituted C₃-C₈ cycloalkyl, or optionally substituted C₃-C₈ heterocyclyl.

—(C═O)NH₂Non-limiting exemplary compounds of the invention are represented in Examples 1 and 2.

As used herein, the term “substituted” or the term “substituent” are related to one or more (e.g. 2, 3, 4, 5, or 6) substituents, wherein each of the one or more substituent(s) is independently selected from hydrogen, halogen, —NO₂, —CN, —OH, oxo, amino, thioxo, carbonyl, imino, —(C═O)NH₂, —CONR′₂, —CNNR′₂, —CSNR′₂, —CONH—OH, —CONH—NH₂, NHCOR′, —NHCSR′, —NHCNR′, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C1-C30 alkyl, optionally substituted C1-C30 alkenyl, optionally substituted C1-C30 alkynyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′2, C₁-C₆ alkyl-SR′, or a combination thereof. In some embodiments, the term “substituted” or the term “substituent” comprises one or more substituents represented by Y, Y′ or by Y″.

As used herein, the term “7-10 ring” is referred to a cyclic aliphatic or aromatic compound comprising between 7 and 10 carbon atoms. In some embodiments, 7-10 ring bicyclic ring comprises between 7 and 8, between 8 and 9, between 9 and 10 carbon atoms including any value therebetween.

As used herein the term “C₁-C₆ alkyl” including any C₁-C₆ alkyl related compounds, is referred to any linear or branched alkyl chain comprising between 1 and 6, between 1 and 2, between 2 and 3, between 3 and 4, between 4 and 5, between 5 and 6, carbon atoms, including any range therebetween. In some embodiments, C₁-C₆ alkyl comprises any of methyl, ethyl, propyl, butyl, pentyl, iso-pentyl, hexyl, and tert-butyl or any combination thereof. In some embodiments, C₁-C₆ alkyl as described herein further comprises an unsaturated bond, wherein the unsaturated bond is located at 1^{st}, 2^{nd}, 3^{rd}, 4^{th}, 5^{th}, or 6^{th} position of the C₁-C₆ alkyl.

As used herein the term “(C₃-C₁₀) cycloalkyl” is referred to an optionally substituted C3, C4, C5, C6, C7, C8, C9 or C10 ring. In some embodiments, (C₃-C₁₀) ring comprises optionally substituted cyclopropane, cyclobutene, cyclopentane, cyclohexane, or cycloheptane.

As used herein the term “(C₃-C₈) cycloalkyl” is referred to an optionally substituted C3, C4, C5, C6, C7, or C8 ring. In some embodiments, (C₃-C₁₀) ring comprises optionally substituted cyclopropane, cyclobutene, cyclopentane, cyclohexane, or cycloheptane.

As used herein the term “(C₆-C₁₂) ring” is referred to an optionally substituted C6, C7, C8, C9, C10, C11, or C12 ring. In some embodiments, (C₆-C₁₂) ring is referred to a bicyclic ring (e.g. fused ring, spirocyclic ring, biaryl ring).

As used herein the term “bicyclic heteroaryl” referred to (C₆-C₁₂) a bicyclic heteroaryl ring, wherein bicyclic (C₆-C₁₀) ring is as described herein.

As used herein the term “bicyclic aryl” referred to (C₆-C₁₂) a bicyclic aryl ring, wherein bicyclic (C₆-C₁₂) ring is as described herein.

As used herein the term “bicyclic heterocyclyl” referred to (C₆-C₁₂) a bicyclic heterocyclic ring, wherein (bicyclic C₆-C₁₂) ring is as described herein.

As used herein the term “bicyclic cycloalkyl” referred to (C₆-C₁₂) a bicyclic cycloalkyl ring, wherein bicyclic (C₆-C₁₂) ring is as described herein.

In some embodiments, the compounds described herein are pharmaceutical pure compounds, having a chemical purity above 95%, above 97%, or above 99%, including any range between.

In some embodiments, the compounds described herein are chiral compounds (i.e. possess an asymmetric carbon atom). In some embodiments, diastereomers, geometric isomers and individual isomers are encompassed within the scope of the present invention. In some embodiments, a chiral compound described herein is in form of a racemic mixture. In some embodiments, a chiral compound is in form of a single enantiomer, with an asymmetric carbon atom having the R configuration. In some embodiments, a chiral compound is in form of a single enantiomer, with an asymmetric carbon atom having the S configuration as described hereinabove.

In some embodiments, a chiral compound is in form of a single enantiomer with enantiomeric purity of more than 70%. In some embodiments, a chiral compound is in form of a single enantiomer with enantiomeric purity of more than 80%. In some embodiments, a chiral compound is in form of a single enantiomer with enantiomeric purity of more than 90%. In some embodiments, a chiral compound is in form of a single enantiomer with enantiomeric purity of more than 95%.

In some embodiments, the compound of the invention comprising an unsaturated bond is in a form of a trans-, or cis-isomer. In some embodiments, the composition of the invention comprises a mixture of cis- and trans-isomers, as described hereinabove.

In some embodiments, the composition of the invention comprises the compound of the invention, a mixture (e.g. racemic mixture) of enantiomers, or is enriched with an enantiomer of interest.

In some embodiments, the compound of the invention comprises any one of the compounds disclosed herein, including any enantiomers thereof. In some embodiments, the compound of the invention comprises a mixture of enantiomers (e.g. a racemic mixture). In some embodiments, the compound of the invention comprises any one of the compounds disclosed herein, including any salt thereof. In some embodiments, the salt of the compound is a pharmaceutically acceptable salt.

### Pharmaceutical Composition

In another aspect of the invention disclosed herein, there is a pharmaceutical composition comprising the compound of the invention, a pharmaceutically acceptable salt thereof or both.

Non-limiting examples of pharmaceutically acceptable salts include but are not limited to: acetate, aspartate, benzenesulfonate, benzoate, bicarbonate, carbonate, halide (such as bromide, chloride, iodide, fluoride), bitartrate, citrate, salicylate, stearate, succinate, sulfate, tartrate, decanoate, edetate, fumarate, gluconate, and lactate or any combination thereof.

In some embodiments, the pharmaceutical composition comprises the compound of the invention and a pharmaceutically acceptable carrier. In some embodiments, the pharmaceutical composition comprises a therapeutically effective amount of the compound of the invention and the pharmaceutically acceptable carrier.

For example, the term “pharmaceutically acceptable” can mean approved by a regulatory agency of the Federal or a state government or listed in the U.S. Pharmacopeia or other generally recognized pharmacopeia for use in animals, and more particularly in humans. In some embodiments, the compound of the invention is referred to herein as an active ingredient of a pharmaceutical composition.

In some embodiments, the pharmaceutical composition as described herein is a topical composition. In some embodiments, the pharmaceutical composition is an oral composition. In some embodiments, the pharmaceutical composition is an injectable composition. In some embodiments, the pharmaceutical composition is for a systemic use.

In some embodiments, the pharmaceutical composition is any of an emulsion, a liquid solution, a gel, a paste, a suspension, a dispersion, an ointment, a cream, or a foam.

As used herein, the term “carrier” refers to a diluent, adjuvant, excipient, or vehicle with which the active ingredient is administered. Such carriers can be sterile liquids, such as water-based and oils, including those of petroleum, animal, vegetable, or synthetic origin, such as peanut oil, soybean oil, mineral oil, sesame oil and the like, polyethylene glycols, glycerin, propylene glycol or other synthetic solvents.

Other non-limiting examples of carriers include, but are not limited to: terpenes derived from Cannabis, or total terpene extract from Cannabis plants, terpenes from coffee or cocoa, mint-extract, eucalyptus-extract, citrus-extract, tobacco-extract, anis-extract, any vegetable oil, peppermint oil, d-limonene, b-myrcene, a-pinene, linalool, anethole, a-bisabolol, camphor, b-caryophyllene and caryophyllene oxide, 1,8-cineole, citral, citronella, delta-3-carene, farnesol, geraniol, indomethacin, isopulegol, linalool, unalyl acetate, b-myrcene, myrcenol, 1-menthol, menthone, menthol and neomenthol, oridonin, a-pinene, diclofenac, nepafenac, bromfenac, phytol, terpineol, terpinen-4-ol, thymol, and thymoquinone. One skilled in the art will appreciate that a particular carrier used within the pharmaceutical composition of the invention may vary depending on the route of administration.

In some embodiments, the carrier improves the stability of the active ingredient in a living organism. In some embodiments, the carrier improves the stability of the active ingredient within the pharmaceutical composition. In some embodiments, the carrier enhances the bioavailability of the active ingredient.

Water may be used as a carrier such as when the active ingredient has a sufficient aqueous solubility, so as to be administered intravenously. Saline solutions and aqueous dextrose and glycerol solutions can also be employed as liquid carriers, particularly for injectable solutions.

In some embodiments, the carrier is a liquid carrier. In some embodiments, the carrier is an aqueous carrier.

Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol, and the like. The composition, if desired, can also contain minor amounts of wetting or emulsifying agents, or pH buffering agents such as acetates, citrates, or phosphates. Antibacterial agents such as benzyl alcohol or methyl parabens; antioxidants such as ascorbic acid or sodium bisulfite; and agents for the adjustment of tonicity such as sodium chloride or dextrose are also envisioned. The carrier may comprise, in total, from 0.1% to 99.99999% by weight of the composition/s or the pharmaceutical composition/s presented herein.

In some embodiments, the pharmaceutical composition includes incorporation of any one of the active ingredients into or onto particulate preparations of polymeric compounds such as polylactic acid, polyglycolic acid, hydrogels, etc., or onto liposomes, microemulsions, micelles, unilamellar or multilamellar vesicles, erythrocyte ghosts, or spheroplasts. Such compositions may influence the physical state, solubility, stability, rate of in vivo release, and rate of in vivo clearance.

In some embodiments, the pharmaceutical composition is a liquid at a temperature between 15 to 45° C. In some embodiments, the pharmaceutical composition is a solid at a temperature between 15 to 45° C. In some embodiments, the pharmaceutical composition is a semi-liquid at a temperature between 15 to 45° C. It should be understood that the term “semi-liquid”, is intended to mean materials which are flowable under pressure and/or shear force. In some embodiments, semi-liquid compositions include creams, ointments, gel-like materials, and other similar materials. In some embodiments, the pharmaceutical composition is a semi-liquid composition, characterized by a viscosity in a range from 31,000-800,000 cps.

Non-limiting examples of carriers for pharmaceutical compositions being in the form of a cream include but are not limited to: non-ionic surfactants (e.g., glyceryl monolinoleate glyceryl monooleate, glyceryl monostearate lanolin alcohols, lecithin mono- and di-glycerides poloxamer polyoxyethylene 50 stearate, and sorbitan trioleate stearic acid), anionic surfactants (e.g. pharmaceutically acceptable salts of fatty acids such as stearic, oleic, palmitic, and lauric acids), cationic surfactants (e.g. pharmaceutically acceptable quaternary ammonium salts such as benzalkonium chloride, benzethonium chloride, and cetylpyridinium chloride) or any combination thereof.

In some embodiments, the pharmaceutical composition being in the form of a cream further comprises a thickener.

Non-limiting examples of thickeners include, but are not limited to microcrystalline cellulose, a starch, a modified starch, gum tragacanth, gelatin, and a polymeric thickener (e.g. polyvinylpyrrolidone) or any combination thereof.

In some embodiments, the pharmaceutical composition comprising the compound of the invention is in a unit dosage form. In some embodiments, the pharmaceutical composition is prepared by any of the methods well known in the art of pharmacy. In some embodiments, the unit dosage form is in the form of a tablet, capsule, lozenge, wafer, patch, ampoule, vial, or pre-filled syringe.

In addition, in vitro assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems. In some embodiments, the effective dose is determined as described hereinabove.

In another embodiment, the pharmaceutical composition of the invention is administered in any conventional oral, parenteral, or transdermal dosage form.

As used herein, the terms “administering”, “administration”, and like terms refer to any method which, in sound medical practice, delivers a composition containing an active agent to a subject in such a manner as to provide a therapeutic effect.

In some embodiments, the pharmaceutical composition is administered via oral (i.e., enteral), rectal, vaginal, topical, sublingual, buccal, nasal, ophthalmic, transdermal, subcutaneous, intramuscular, intraperitoneal, intrathecal, or intravenous routes of administration. The route of administration of the pharmaceutical composition will depend on the disease or condition to be treated. Suitable routes of administration include, but are not limited to, parenteral injections, e.g., intradermal, intravenous, intramuscular, intralesional, subcutaneous, intrathecal, and any other mode of injection as known in the art. In addition, it may be desirable to introduce the pharmaceutical composition of the invention by any suitable route, including intraventricular and intrathecal injection; intraventricular injection may be facilitated by an intraventricular catheter, for example, attached to a reservoir. Pulmonary administration can also be employed, e.g., by use of an inhaler or nebulizer.

In some embodiments, the pharmaceutical composition is in a form of, for example, and not by way of limitation, an ointment, cream, gel, paste, foam, aerosol, suppository, pad, or gelled stick.

In some embodiments, for oral applications, the pharmaceutical composition is in the form of a tablet or a capsule, which can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate; or a glidant such as colloidal silicon dioxide. When the dosage unit form is a capsule, it can contain, in addition to materials of the above type, a liquid carrier such as fatty oil. In addition, dosage unit forms can contain various other materials which modify the physical form of the dosage unit, for example, coatings of sugar, shellac, or other enteric agents. In some embodiments, the tablet of the invention is further film coated. In some embodiments, oral application of the pharmaceutical composition or of the kit is in a form of a drinkable liquid. In some embodiments, oral application of the pharmaceutical composition or of the kit is in a form of an edible product.

For purposes of parenteral administration, solutions in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous, and intraperitoneal injection purposes.

In some embodiments, the pharmaceutical composition is for use in the inhibition of protein kinase-R (PKR). In some embodiments, inhibition of PKR comprises inhibition of cellular activity of PKR, wherein inhibition is as described herein. In some embodiments, inhibition comprises a selective inhibition of PKR activity.

In some embodiments, the compound has at least 2 times, at least 5 times, at least 10 times, at least 15 times, at least 20 times, at least 30 times, at least 30 times, at least 50 times, at least 80 times, at least 100 times, at least 200 times, at least 300 times, at least 400 times, at least 500 times, at least 700 times, at least 1000 times, at least 10,000 times, at least 50,000 times, at least 100,000 times lower IC₅₀ for the PKR as compared to other eIF2alpha phosphorylation kinases (e.g. PKR-like ER kinase (PERK), general control non-derepressible-2 (GCN2) or heme-regulated inhibitor (HRI)). In some embodiments, the compound is substantially devoid of inhibitory activity with respect to non-PKR kinases (e.g. eIF2alpha phosphorylation kinases).

In some embodiments, compounds of the invention (e.g. represented in the Example 1) inhibit 50% of PKR activity at a concentration (e.g. IC50) of less than 10000 nM, less than 5000 nM, less than 2000 nM, less than 1000 nM, less than 200 nM, less than 500 nM, less than 50 nM, less than 10 nM, less than 5 nM, less than 1 nM. In some embodiments, inhibition of PKR activity is evaluated in-vitro (e.g. in a cell-free biochemical assay, as represented in the Examples section).

In some embodiments, the pharmaceutical composition is for use in the prevention or inhibition of PKR associated cell toxicity. In some embodiments, the pharmaceutical composition is for use in the prevention or inhibition of eIF2alpha phosphorylation. In some embodiments, the pharmaceutical composition is for use in the prevention or inhibition of TAU phosphorylation. In some embodiments, the pharmaceutical composition is for use in the prevention or inhibition of PKR associated cell toxicity. In some embodiments, the pharmaceutical composition is for use in the inhibition of PKR. In some embodiments, the pharmaceutical composition is for use in inhibition of eIF2alpha phosphorylation. In some embodiments, the pharmaceutical composition is for use in the inhibition of TAU phosphorylation (directly, via GSK3β or other). In some embodiments, the pharmaceutical composition is for use in the prevention of cell cytotoxicity. In some embodiments, the pharmaceutical composition is for use in the initiation or enhancement of an anti-inflammatory effect via PKR inhibition (e.g. inhibition of NfKB pathway, and/or inhibition of inflammasome activation). In some embodiments, the pharmaceutical composition is for use in the reduction of amyloid-beta aggregates, wherein amyloid-beta aggregates are as described herein.

In some embodiments, the cell is a nerve cell. In some embodiments, the cell is a neuron. In some embodiments, the cell is a glia cell. In some embodiments, the cell is an immune cell. In some embodiments, the cell is a cancerous cell. In some embodiments, the cell expresses PKR. In some embodiments, the cell is characterized by an elevated expression of PKR. In some embodiments, the cell is characterized by an elevated activation of PKR.

In some embodiments, the cell is a cancer cell. In some embodiments, cancer is a PKR related cancer. In some embodiments, cancer is related to an abnormal expression of PKR.

In some embodiments, the cancer cell is selected from the group comprising a breast cancer cell, a colon cancer cell, a skin cancer cell, a hematopoietic cancer cell, or any combination thereof.

In some embodiments, the pharmaceutical composition is for use in the prevention or treatment of abnormal cell growth and/or metastasis of a cell expressing PKR in a subject in need thereof. In some embodiments, the pharmaceutical composition is for use in the prevention or treatment of abnormal proliferation of cells expressing PKR.

In some embodiments, the pharmaceutical composition is for use in the prevention or treatment of a neurodegenerative disorder, a neuroinflammatory disorder, a metabolic disease, a cardiovascular disease, a proliferative disease, or any combination thereof.

In some embodiments, the pharmaceutical composition is for use in the prevention or treatment of a cognitive disorder. In some embodiments, the pharmaceutical composition is for use in the amelioration of a condition associated with a cognitive disorder.

In some embodiments, the pharmaceutical composition is for use in the prevention or treatment of hypoxia (e.g. of a muscle tissue, such as cardiac muscle tissue) in a subject in need thereof. In some embodiments, the pharmaceutical composition is for use in the prevention or treatment of ischemia (e.g. of a muscle tissue, such as cardiac muscle tissue) in a subject in need thereof.

### Method

In another aspect, there provided herein is a method for preventing or treating a disease or a disorder, or ameliorating a condition associated with an abnormal or normal activity of PKR in a subject, comprising administering to the subject the pharmaceutical composition of the invention, thereby (i) preventing or treating a disease or a disorder; or (ii) ameliorating the condition associated with an abnormal or normal expression of PKR and/or activation in a subject. In another aspect, there provided herein is a method for preventing or treating a disease or a disorder, or ameliorating a condition associated with an abnormal or normal activity of PKR in a subject, comprising administering to the subject the pharmaceutical composition of the invention, thereby preventing or treating a disease or a disorder. In some embodiments, there is a method is for preventing or treating a disease or a disorder comprising a neurodegenerative disorder, a neuroinflammatory disorder, a metabolic disease, a cardiovascular disease, a proliferative disease, or any combination thereof.

In some embodiments, there is a method is for preventing or treating cancer (e.g. cancer associated with abnormal or normal PKR activity) comprising administering to the subject the pharmaceutical composition or the compound of the invention. In some embodiments, the method is for preventing or treating cancer characterized by a cell expressing PKR. In some embodiments, the method is for prevention or inhibition of cell mitosis, wherein the cell is as described hereinabove.

In some embodiments, the method comprises administering a compound as described herein to a subject afflicted with cancer. In some embodiments, the method comprises administering a compound as described herein to a subject afflicted with cancer characterized by cancerous cells that express PKR and/or highly express PKR. In some embodiments, the method comprises administering a compound as described herein to a subject afflicted with cancer characterized by cancerous cells that express activated PKR. In some embodiments, the method further comprises selecting a subject afflicted with cancer, wherein the subject comprises a cancerous cell expressing PKR and/or highly expressing PKR.

In some embodiments, administering is by an oral administration, a topical administration, a systemic administration, or a combination thereof.

In some embodiments, the neurological disorder to be treated with a compound and/or a composition as described herein, is selected from: multiple sclerosis, Alzheimer's disease, dementia, Parkinson's disease, Huntington's disease, Down syndrome, Amyotrophic lateral sclerosis (ALS), frontotemporal dementia (FTD), Dystonia and prion disease or any combination thereof and acquired and inherited neuropathies in the peripheral nervous system. In some embodiments, the neurological disorder is multiple sclerosis. In some embodiments, the neurological disorder is Alzheimer's disease.

In some embodiments, the method comprises ameliorating a condition associated with an abnormal or normal activity of PKR in a subject comprises, wherein ameliorating comprises (i) improving cognitive function, or (ii) inhibiting cognitive dysfunction of the subject.

As used herein, the term “cognitive function” is well-known in the art and refers to multiple mental abilities, including learning, thinking, reasoning, remembering, problem solving, decision making, and attention.

In some embodiments, the method is for inducing neuroprotective effect of a nerve cell and/or glia cell of the subject.

In some embodiments, the method is for preventing or reducing inflammation of a nerve cell and/or glia cell of the subject. In some embodiments, the method is for preventing or reducing inflammation of a nervous tissue.

In some embodiments, the method is for preventing or reducing apoptosis of a nerve cell and/or glia cell of the subject.

In some embodiments, the method is for preventing or reducing accumulation of amyloid-beta aggregates within a subject in need thereof. In some embodiments, the amyloid-beta aggregates comprise intracellular aggregates, extracellular aggregates, aggregates within a nerve tissue, and aggregates within a nervous system or any combination thereof. In some embodiments, the intracellular and/or extracellular aggregates refer to a cell selected from a nerve cell, a glia cell, a cancer cell, epithelial cell or a muscle cell or any combination thereof. In some embodiments, the method is for preventing or reducing accumulation of amyloid-beta aggregates within a nervous tissue.

In some embodiments, the method is for preventing or reducing accumulation of tau-protein aggregates. In some embodiments, the tau-protein aggregates are within a nerve cell and/or a glia cell of the subject. In some embodiments, the tau-protein aggregates are within a nerve tissue, and/or within a nervous system of the subject.

In some embodiments, the method is for preventing or treating frontotemporal dementia, frontotemporal lobar degeneration. In some embodiments, the method is for preventing or reducing a tauopathy selected from corticobasal degeneration, frontotemporal dementia, frontotemporal lobar degeneration, progressive supranuclear palsy, and Pick's disease, including any combination thereof.

In some embodiments, the method is for preventing or treating a disease or disorder associated with an abnormal or normal accumulation of amyloid-beta aggregates, wherein the amyloid-beta aggregates are as described herein. In some embodiments, the method is for preventing or treating a disease or disorder associated with an abnormal or normal accumulation of tau protein. In some embodiments, the accumulation of tau protein is extracellular and/or intracellular accumulation. In some embodiments, the accumulation of tau protein is within a cell and/or a tissue of the subject.

Treatment effectiveness and identification of a subject that can benefit from a compound and/or a composition as describe herein, can be monitored/identified by methods which include MRI, PET, PET-CT, etc. Treatment effectiveness can be evaluated by monitoring physiological parameters and/or disease related biomarkers of the subject. Such physiological parameters are well-known in the art.

In some embodiments, the method comprises administering the pharmaceutical composition of the invention at least 1 time, at least 2 times, at least 3 times, at least 4 times, at least 5 times, at least 7 times, or at least 10 times per day or per week or per month, or any value and range therebetween. Each possibility represents a separate embodiment of the invention. In some embodiments, the method comprises administering the composition or the combination of the invention 1-2 times per day or per week or per month, 1-3 times per day or per week or per month, 1-4 times per day or per week or per month, 1-5 times per day, 1-7 times per day or per week or per month, 2-3 times per day or per week or per month, 2-4 times per day or per week or per month, 2-5 times per day or per week or per month, 3-4 times per day or per week or per month, 3-5 times per day or per week or per month, or 5-7 times per day or per week or per month. Each possibility represents a separate embodiment of the invention.

In some embodiments, the method comprises administering the pharmaceutical composition of the invention to the subject at a daily or weekly or monthly dosage of 0.05 to 20 mg/kg, 0.05 to 0.1 mg/kg, 0.1 to 0.3 mg/kg, 0.3 to 0.5 mg/kg, 0.5 to 0.8 mg/kg, 0.8 to 1 mg/kg, 1 to 2 mg/kg, 2 to 5 mg/kg, 5 to 10 mg/kg, 10 to 15 mg/kg, 15 to 20 mg/kg including any range or value therebetween.

It should be apparent to one skilled in the art, that for example in-vitro and in-vivo assays may optionally be employed to help identify optimal dosage ranges. The precise dose to be employed in the formulation will also depend on the route of administration, and the nature of the disease or disorder, and should be decided according to the judgment of the practitioner and each patient's circumstances. Effective doses can be extrapolated from dose-response curves derived from in-vitro or in-vivo animal model test bioassays or systems.

In some embodiments, the subject is a mammal. In some embodiments, the subject is a lab animal. In some embodiments, the subject is a pet. In some embodiments, the subject is a rodent. In some embodiments, the subject is a farm animal. In some embodiments, the subject is a human subject.

In some embodiments, the composition of the present invention is administered in a therapeutically safe and effective amount. As used herein, the term “safe and effective amount” refers to the quantity of a component which is sufficient to yield a desired therapeutic response without undue adverse side effects, including but not limited to toxicity, such as calcemic toxicity, irritation, or allergic response, commensurate with a reasonable benefit/risk ratio when used in the presently described manner. The actual amount administered, and the rate and time-course of administration, will depend on the nature and severity of the condition being treated. Prescription of treatment, e.g. decisions on dosage, timing, etc., is within the responsibility of general practitioners or specialists, and typically takes account of the disorder to be treated, the condition of the individual patient, the site of delivery, the method of administration and other factors known to practitioners. Examples of techniques and protocols can be found in Remington: The Science and Practice of Pharmacy, 21 st Ed., Lippincott Williams & Wilkins, Philadelphia, Pa., (2005).

In some embodiments, the effective amount or dose of the active ingredient can be estimated initially from in vitro assays. In one embodiment, a dose can be formulated in animal models and such information can be used to determine useful doses more accurately in humans.

In one embodiment, toxicity and therapeutic efficacy of the active ingredients described herein can be determined by standard pharmaceutical procedures in vitro, in cell cultures or experimental animals. In one embodiment, the data obtained from these in vitro and cell culture assays and animal studies can be used in formulating a range of dosage for use in human. In one embodiment, the dosages may vary depending on the dosage form employed and the route of administration utilized. In one embodiment, the exact formulation, route of administration and dosage can be chosen by the individual physician in view of the patient's condition. [See e.g., Goodman and Gilman's The Pharmacological Basis of Therapeutics, 13th Ed., McGraw-Hill/Education, New York, NY (2017)].

In some embodiments, the subject is afflicted with a disease or disorder associated with an or normal PKR expression and/or activation. In some embodiments, the subject is afflicted with a disease or disorder selected from the group comprising: breast cancer, colon cancer, skin cancer, basal-cell carcinoma, leukemia, lymphoma.

In some embodiments, the subject is afflicted with a neurological disease or disorder selected from the group comprising: multiple sclerosis, Alzheimer's Disease, multiple sclerosis, dementia, Parkinson's disease, Huntington's disease, Down syndrome, Amyotrophic lateral sclerosis (ALS), frontotemporal dementia (FTD), Dystonia and prion disease or any combination thereof and acquired and inherited neuropathies in the peripheral nervous system. In some embodiments, the neurological disorder is multiple sclerosis. In some embodiments, the neurological disorder is frontotemporal dementia, frontotemporal lobar degeneration and/or any other tauopathy, as described herein.

In some embodiments, the method is for reducing or inhibiting: abnormal cell proliferation, tumor growth, malignancy, or any combination thereof in a subject in need thereof. In some embodiments, the method is for reducing or inhibiting proliferation of cells expressing PKR. In some embodiments, the method is for selectively reducing or inhibiting proliferation of cells expressing PKR.

In some embodiments, reducing comprises at least 50%, at least 60%, at least 70%, at least 80%, at least 85%, at least 90%, at least 92%, at least 95%, at least 97%, at least 99% reduction of the cell proliferation, including any value therebetween.

In some embodiments, reducing comprises at least 10%, 20%, 30%, 40%, 50%, at least 60%, at least 70%, at least 80%, at least 85%, at least 90%, at least 92%, at least 95%, at least 97%, at least 99% reduction of PKR activity, including any value therebetween.

In some embodiments, the compound of the invention has ICso in inhibiting PKR activity between 0.1 and 1 nM, between 1 and 5 nM, between 5 and 10 nM, between 10 and 50 nM, between 50 and 100 nM, between 100 and 500 nM, between 500 and 1 uM, between 1 and 5 uM, between 5 and 10 uM, including any value therebetween.

In some embodiments, the compound has at least 2 times, 5 times, at least 10 times, at least 15 times, at least 20 times, at least 30 times, at least 30 times, at least 50 times, at least 80 times, at least 100 times, at least 200 times, at least 300 times, at least 400 times, at least 500 times, at least 700 times, at least 1000 times, at least 10,000 times, at least 50,000 times, at least 100,000 times lower IC₅₀ for PKR as compared to other eIF2alpha phosphorylation kinases (e.g. PKR-like ER kinase (PERK), general control non-derepressible-2 (GCN2) or heme-regulated inhibitor (HRI)).

### Definitions

The term “one or more” refers to any numerical value selected form of 1, 2, 3, 4, 5, or 6.

As used herein, the term “alkyl” describes an aliphatic hydrocarbon including straight chain and branched chain groups. The alkyl group has between 1 and 20, between 1 and 10, between 1 and 15, between 1 and 5, between 1 and 3, between 2 and 20, between 2 and 10, between 2 and 5, or 1, 2, 3, 4, 5, or 6 carbon atoms, including any range or value between. Whenever a numerical range e.g., “1-20”, is stated herein, it implies that the group, in this case the alkyl group, may contain 1 carbon atom, 2 carbon atoms, 3 carbon atoms, etc., up to and including 20 carbon atoms. The alkyl can be substituted or unsubstituted, as defined herein. The “alkyl” further encompasses a “heteroalkyl”, i.e. an alkyl including one or more heteroatoms (e.g. O, S, N, or NH) within the backbone of the alkyl chain.

The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e. rings which share an adjacent pair of carbon atoms) group where one or more of the rings does not have a completely conjugated pi-electron system. The cycloalkyl group may be substituted or unsubstituted, saturated or containing at least one unsaturated bond, as indicated herein.

The term “cyclyl” describes an aryl, a polycyclyl, a heteroaryl, a cycloalkyl, or heterocyclyl or any combinations thereof.
the term “polycyclic ring” or “polycyclyl” encompasses a plurality (e.g., 2, 3, 4, 5 or 6) of fused or adjacent rings (e.g., biaryl or bicyclohexyl), wherein each ring is independently selected from aryl, heteroaryl, an optionally unsaturated cycloalkyl, an optionally unsaturated heterocyclyl, or any combination thereof. In some embodiments, the term “polycyclyl” encompasses a polycyclic aromatic ring, a polycyclic aliphatic ring, or a mixed polycyclic ring. The term “polycyclic ring” or “polycyclyl” encompasses fused rings (e.g. fused aliphatic, fused aromatic and/or heteroaromatic ring), spirocyclic rings, bridged rings, a dicyclyl (two aromatic and/or aliphatic rings joined by a single carbon-carbon bond), such as presented below:

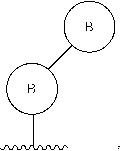

wherein each B independently comprises any of: (i) an optionally substituted aliphatic ring, optionally comprising one or more heteroatoms, (ii) an optionally substituted aromatic or heteroaromatic ring, (iii) an optionally substituted bicyclic aliphatic, bicyclic aromatic/heteroaromatic, or a bicyclic mixed aromatic/heteroaromatic-aliphatic ring, wherein each of the rings optionally comprises one or more heteroatom(s); (iv) a polycyclic aromatic, polycyclic heteroaromatic, or polycyclic aliphatic ring, or a mixed polycyclic ring.

The term “mixed polycyclic ring” refers to any plurality of rings covalently bound to each other (e.g., fused rings, dicylyls, spirocyclic rings etc.) comprising at least one aromatic ring (aryl, or heteroaryl) and at least one aliphatic or non-aromatic ring (optionally a heterocyclyl and/or unsaturated cyclyl).

the term “bicyclic ring” encompasses a fused ring (fused aromatic and/or heteroaromatic ring), spirocyclic ring, a bridged ring, a dicyclyl (two aromatic and/or aliphatic rings joined by a single carbon-carbon bond). The terms “bicyclyl” and “bicyclic ring” are used herein interchangeably.

The term “aryl” describes an all-carbon monocyclic or fused-ring polycyclic (i.e. rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein.

The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, —NH—, oxygen, and sulfur. The rings may also have one or more double bonds. However, the rings do not have a completely conjugated pi-electron system. Representative examples are piperidine, piperazine, tetrahydrofuran, tetrahydropyran, morpholino and the like.

The term “heteroatom” describes O, S, N, NH, NH2, or N(R′)1-2 as allowed by valency.

The term “carboxy” or “carboxylate” describes a —C(O)OR′ group, where R′ is hydrogen, alkyl, cycloalkyl, alkenyl, aryl, heteroaryl (bonded through a ring carbon) or heterocyclyl (bonded through a ring carbon) as defined herein. The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein.

The term “carbonyl” describes a —C(O)R′ group, where R′ is as defined hereinabove.

The above-terms also encompass thio-derivatives thereof (thiocarboxy and thiocarbonyl).

The term “thiocarbonyl” describes a —C(S)R′ group, where R′ is as defined hereinabove.

A “thiocarboxy” group describes a —C(S)OR′ group, where R′ is as defined herein.

A “sulfinyl” group describes an —S(O)R′ group, where R′ is as defined herein.

A “sulfonyl” or “sulfonate” group describes an —S(O)₂R′ group, where R′ is as defined herein.

A “carbamyl” or “carbamate” group describes an —OC(O)NR′R″ group, where R′ is as defined herein and R″ is as defined for R′.

A “nitro” group refers to a —NO2 group.

The term “amide” as used herein encompasses C-amide and N-amide.

The term “C-amide” describes a —C(O)NR′R″ end group or a —C(O)NR′-linking group, as these phrases are defined hereinabove, where R′ and R″ are as defined herein.

The term “N-amide” describes a —NR″C(O)R′ end group or a —NR′C(O)— linking group, as these phrases are defined hereinabove, where R′ and R″ are as defined herein.

The term “carboxylic acid derivative” as used herein encompasses carboxy, amide, carbonyl, anhydride, carbonate ester, and carbamate.

A “cyano” or “nitrile” group refers to a —CN group.

The term “azo” or “diazo” describes an —N═NR′ end group or an —N═N— linking group, as these phrases are defined hereinabove, with R′ as defined hereinabove.

The term “guanidine” describes a —R′NC(N)NR″R′″ end group or a —R′NC(N) NR″— linking group, as these phrases are defined hereinabove, where R′, R″ and R′″ are as defined herein.

As used herein, the term “azide” refers to a —N3 group.

The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove.

The term “phosphinyl” describes a —PR′R″ group, with R′ and R″ as defined hereinabove.

The term “alkylaryl” describes an alkyl, as defined herein, which is substituted by an aryl, as described herein. An exemplary alkylaryl is benzyl.

The term “heteroaryl” describes a monocyclic (e.g. C₅-C₆ heteroaryl ring) or fused ring (i.e. rings which share an adjacent pair of atoms) group having in the ring(s) one or more atoms, such as, for example, nitrogen, oxygen, and sulfur and, in addition, having a completely conjugated pi-electron system. In some embodiments, the terms “heteroaryl” and “C₅-C₆ heteroaryl” are used herein interchangeably. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted by one or more substituents, as described hereinabove. Representative examples are thiadiazol, pyridine, pyrrole, oxazole, indole, purine, and the like. In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydro-isoquinolinyl, chromonyl, 3,4-dihydroiso-quinoxalinyl, 4-(3H)quinazolinonyl, 4H-chromenyl, 4-chromanonyl, oxindolyl, 1,2,3,4-tetrahydroisoquinolinyl, 1,2,3,4-tetrahydro-quinolinyl, 1H-2,3-dihydroisoindolyl, 2,3-dihydrobenzo[f]isoindolyl, 1,2,3,4-tetrahydrobenzo-[g]isoquinolinyl, 1,2,3,4-tetrahydro-benzo[g]isoquinolinyl, chromanyl, isochromanonyl, 2,3-dihydrochromonyl, 1,4-benzo-dioxanyl, 1,2,3,4-tetrahydro-quinoxalinyl, 5,6-dihydro-quinolyl, 5,6-dihydroiso-quinolyl, 5,6-dihydroquinoxalinyl, 5,6-dihydroquinazolinyl, 4,5-dihydro-1H-benzimidazolyl, 4,5-dihydro-benzoxazolyl, 1,4-naphthoquinolyl, 5,6,7,8-tetrahydro-quinolinyl, 5,6,7,8-tetrahydro-isoquinolyl, 5,6,7,8-tetrahydroquinoxalinyl, 5,6,7,8-tetrahydroquinazolyl, 4,5,6,7-tetrahydro-1H-benzimidazolyl, 4,5,6,7-tetrahydro-benzoxazolyl, 1H-4-oxa-1,5-diaza-naphthalen-2-onyl, 1,3-dihydroimidizolo-[4,5]-pyridin-2-onyl, 2,3-dihydro-1,4-dinaphtho-quinonyl, 2,3-dihydro-1H-pyrrol[3,4-b]quinolinyl, 1,2,3,4-tetrahydrobenzo[b]-[1,7]naphthyridinyl, 1,2,3,4-tetra-hydrobenz[b][1,6]-naphthyridinyl, 1,2,3,4-tetrahydro-9H-pyrido[3,4-b]indolyl, 1,2,3,4-tetrahydro-9H-pyrido[4,3-b]indolyl, 2,3-dihydro-1H-pyrrolo-[3,4-b]indolyl, 1H-2,3,4,5-tetrahydro-azepino[3,4-b]indolyl, 1H-2,3,4,5-tetrahydroazepino-[4,3-b]indolyl, 1H-2,3,4,5-tetrahydro-azepino[4,5-b]indolyl, 5,6,7,8-tetrahydro[1,7]napthyridinyl, 1,2,3,4-tetrahydro-[2,7]-naphthyridyl, 2,3-dihydro[1,4]dioxino[2,3-b]pyridyl, 2,3-dihydro[1,4]-dioxino[2,3-b]79escry79, 3,4-dihydro-2H-1-oxa[4,6]diazanaphthalenyl, 4,5,6,7-tetrahydro-3H-imidazo-[4,5-c]pyridyl, 6,7-dihydro[5,8]diazanaphthalenyl, 1,2,3,4-tetrahydro[1,5]-napthyridinyl, 1,2,3,4-tetrahydro[1,6]napthyridinyl, 1,2,3,4-tetrahydro[1,7]napthyridinyl, 1,2,3,4-tetrahydro-[1,8]napthyridinyl or 1,2,3,4-tetrahydro[2,6]napthyridinyl. In some embodiments, heteroaryl groups are optionally substituted. In one embodiment, the one or more substituents are each independently selected from among halo, hydroxy, amino, cyano, nitro, alkylamido, acyl, C1-6-alkyl, C1-6-haloalkyl, C1-6-hydroxyalkyl, C1-6-aminoalkyl, C1-6-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, oxadiazole, benzopyrazole, quinolizine, cinnoline, phthalazine, quinazoline and quinoxaline. In some embodiments, the substituents are halo, hydroxy, cyano, 0-C₁-6-alkyl, C₁₋₆-alkyl, hydroxy-C₁₋₆-alkyl and amino-C₁₋₆-alkyl.

As used herein, the terms “halo” and “halide”, which are referred to herein interchangeably, describe an atom of a halogen, that is fluorine, chlorine, bromine, or iodine, also referred to herein as fluoride, chloride, bromide, and iodide.

The term “haloalkyl” describes an alkyl group as defined above, further substituted by one or more halide(s).

Examples of isotopes that can be incorporated into compounds of the present disclosure include isotopes of hydrogen, carbon, nitrogen, oxygen, phosphorous, fluorine, and chlorine, such as 2H, 3H, 11C, 13C, 15N, 17O, 18O, 18F, 31P, 32P, 35S, 36Cl, and 125I, respectively. In one embodiment, isotopically labeled compounds can be used in metabolic studies (with 14C), reaction kinetic studies (with, for example 2H or 3H), detection or imaging techniques, such as positron emission tomography (PET) or single-photon emission computed tomography (SPECT) including drug and substrate tissue distribution assays, or in radioactive treatment of patients. In particular, an 18F labeled compound may be particularly desirable for PET or SPECT studies. Isotopically labeled compounds of this invention and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (D) and tritium (3H) may optionally be used anywhere in described structures that achieves the desired result. Alternatively or in addition, isotopes of carbon, e.g., 13C and 14C, may be used. In one embodiment, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tmax, Cmax, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain embodiments, the isotope is 80, 85, 90, 95, or 99% or more enriched in an isotope at any location of interest. In some embodiments, deuterium is 80, 85, 90, 95, or 99% enriched at a desired location. Unless otherwise stated, the enrichment at any point is above natural abundance, and in an embodiment is enough to alter a detectable property of the compounds as a drug in a human.

The compounds of the present disclosure may form a solvate with solvents (including water). Therefore, in one embodiment, the invention includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with the invention include those wherein the solvent of crystallization may be isotopically substituted, e.g., D20, d6-acetone, or d6-DMSO. A solvate can be in a liquid or solid form.

### General

As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

As used herein, the term “prevention” of a disease, disorder, or condition encompasses the delay, prevention, suppression, or inhibition of the onset of a disease, disorder, or condition. As used in accordance with the presently described subject matter, the term “prevention” relates to a process of prophylaxis in which a subject is exposed to the presently described active ingredients prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of inflammatory disorders.

The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression.

In some embodiments, the term “reducing”, or “enhancing” including any grammatical from thereof relates to at least 50%, at least 60%, at least 70%, at least 80%, at least 85%, at least 90%, at least 92%, at least 95%, at least 97%, at least 99% reduction/enhancement (e.g. of enzymatic activity), compared to a control or a baseline, including any range between.

Conversely, the term “treatment” refers to the clinical application of active agents to combat an already existing condition whose clinical presentation has already been realized in a patient.

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an” and “at least one” are used interchangeably in this application.

As used herein the term “about” refers to ±10%.

The terms “comprises”, “comprising”, “includes”, “including”, “having” and their conjugates mean “including but not limited to”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise”, “include”, and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise” or variations such as “comprises” or “comprising” indicate the inclusion of any recited integer or group of integers but not the exclusion of any other integer or group of integers.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure, or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises” “comprising”, and “having” are/is interchangeable with “consisting”.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent, or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

### EXAMPLES

Generally, the nomenclature used herein, and the laboratory procedures utilized in the present invention include molecular, biochemical, and microbiological techniques. Such techniques are thoroughly explained in the literature.

### Example 1

### In-Vitro PKR Inhibitors

Several compounds of the invention showed significant PKR inhibition in-vitro, showing micromolar and sub-micromolar IC₅₀ values. Some compound exhibited IC₅₀ values between 1 and 10 nM (e.g. in a cell-free biochemical assay).

Specific representative examples of potent PKR inhibitors of Formula VII:

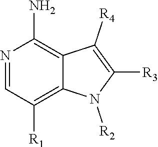

are represented in Table 1 below. Additional potent PKR compounds of the invention have been synthesized by the inventors.

TABLE 1
Exemplary PKR inhibitors represented by Formula VII

| R1 | R2 | R3 | R4 |
|---|---|---|---|
| NC— | 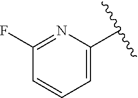 [SMILES: *c1cccc(F)n1] | — | 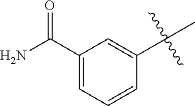 [SMILES: *c1cccc(C(N)=O)c1] |
| NC— | 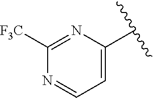 [SMILES: *c1ccnc(C(F)(F)F)n1] | — | 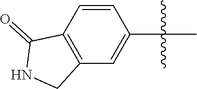 [SMILES: *c1ccc2c(c1)CNC2=O] |
| NC— | 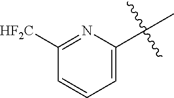 | — | 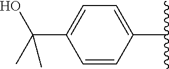 [SMILES: *c1ccc(C(C)(C)O)cc1] |
| NC— | 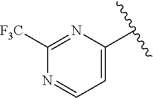 [SMILES: *c1ccnc(C(F)(F)F)n1] | — | 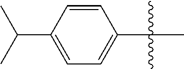 [SMILES: *c1ccc(C(C)C)cc1] |
| 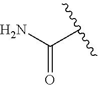 [SMILES: *C(N)=O] | 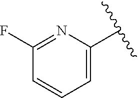 [SMILES: *c1cccc(F)n1] | — | 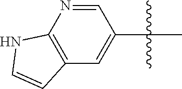 [SMILES: *c1cnc2[nH]ccc2c1] |
| NC— | — | — | 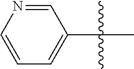 [SMILES: *c1cccnc1] |
| 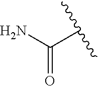 [SMILES: *C(N)=O] | — | 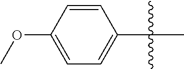 [SMILES: *c1ccc(OC)cc1] | I— |
| NC— | — | 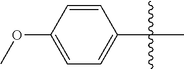 [SMILES: *c1ccc(OC)cc1] | 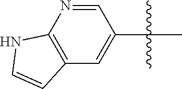 [SMILES: *c1cnc2[nH]ccc2c1] |

Additionally, compounds of Formula VII with R3 being pyridine, and alternatively with R2 being a pyridine substituted by electron-donating groups, and/or substituted aromatic rings, heteroaromatic substituted/unsubstituted rings, fused heteroaromatic rings in the R4 position were also potent PKR inhibitors.

### Example 2

### In-Vitro PKR Inhibitors

Additional exemplary compounds of the invention showed significant PKR inhibition in-vitro, showing micromolar and sub-micromolar IC₅₀ values. Further to the compounds disclosed in the Example 1, the inventors synthesized about 120 additional compounds which showed significant PKR inhibition in-vitro.

Non-limiting examples of additional PKR inhibitors represented by Formula VII:

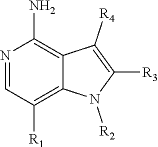

### or by Formula VIIA:

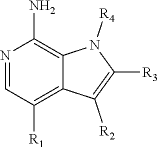

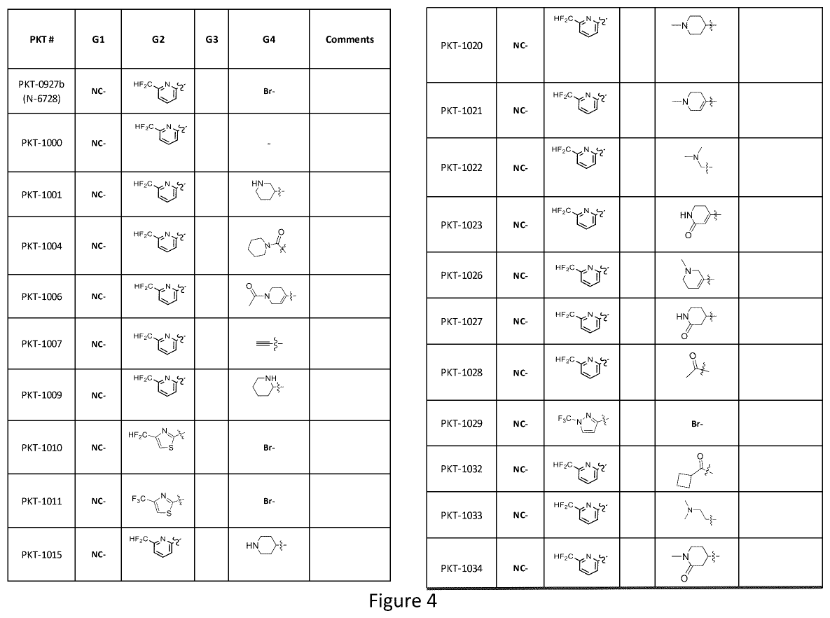

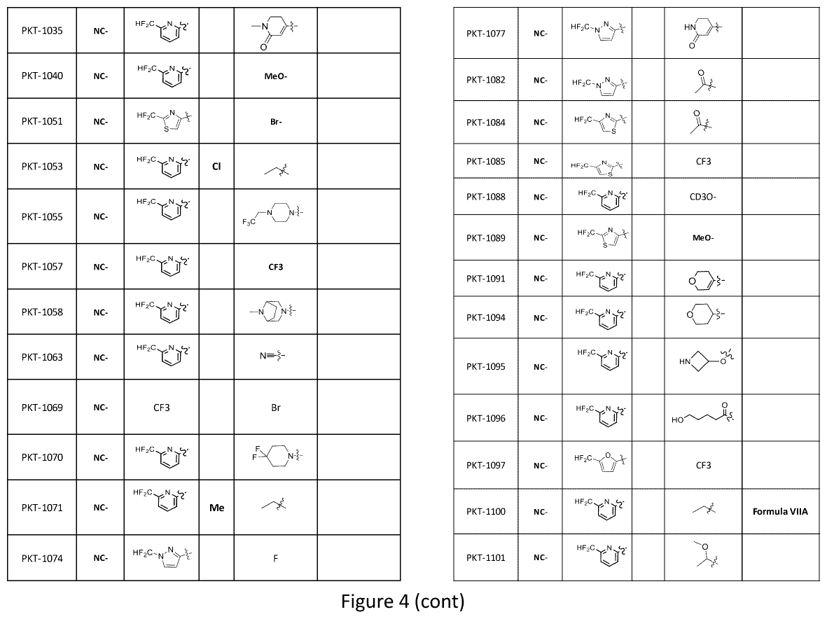

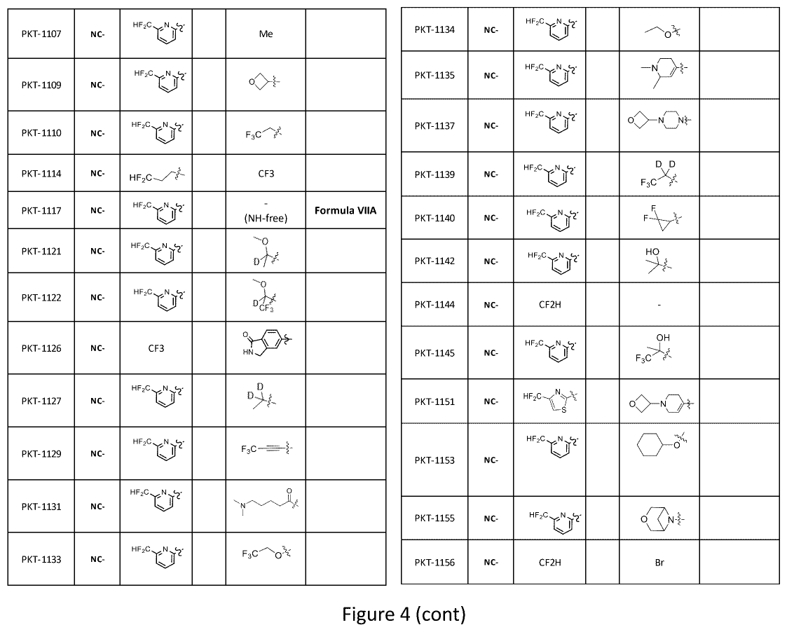

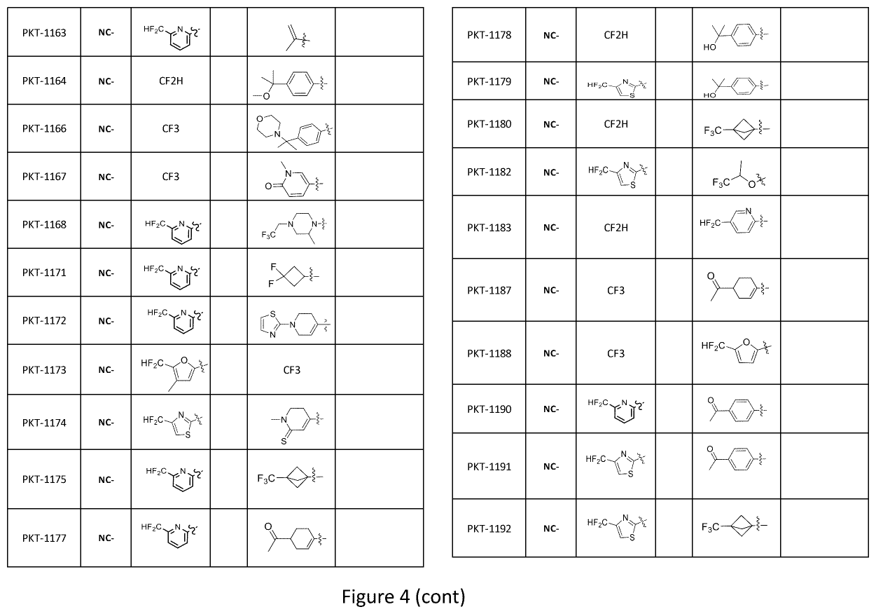

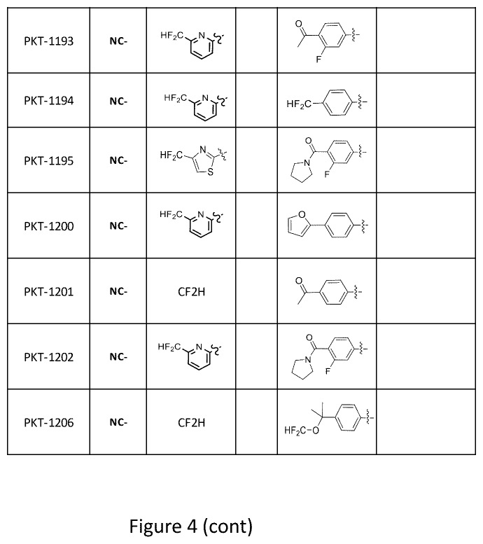

are represented in FIG. 4.

Some of the compounds presented in FIG. 4 were tested in a cell-based assay and showed a significant cell-based potency.

Exemplary cell-based assay is as follows: The assay used to determine the cellular activity of the compounds is an In-Cell Western (ICW) assay, which is a cellular quantitative immunofluorescence assay that measures the level of p-PKR in a human cell-line. This assay is performed in 384-well plates and the level of p-PKR is detected following induction of PKR pathway using Poly I:C, with or without the tested compounds at several concentrations. The generated data is used to plot the dose response inhibition curve and calculate the IC50 of the tested compounds.

Surprisingly, closely related compounds which are not according to the invention, showed a negligible PKR inhibition (IC50 above 1 uM) in the cell-free enzymatic assay.

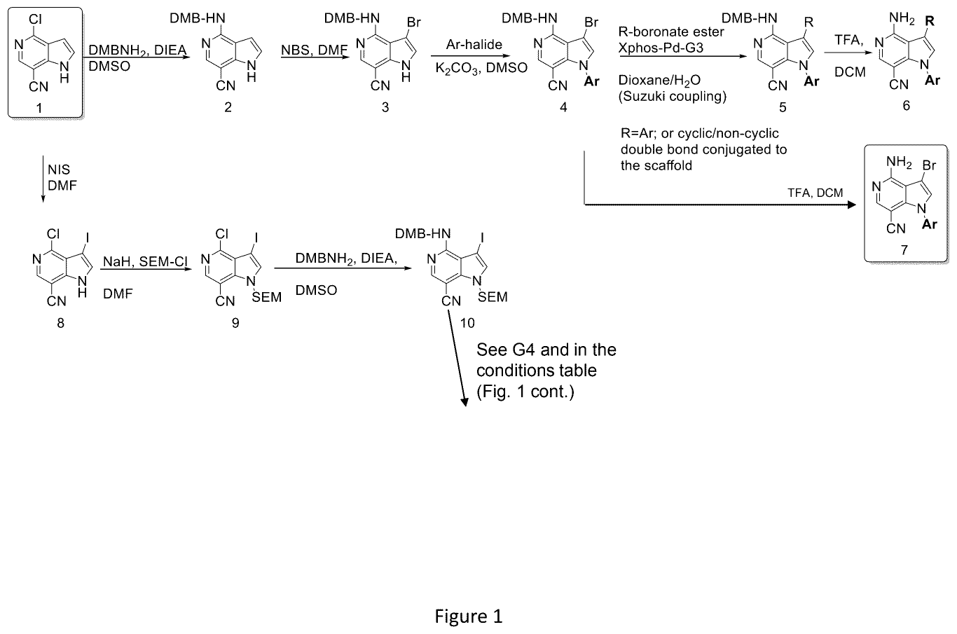

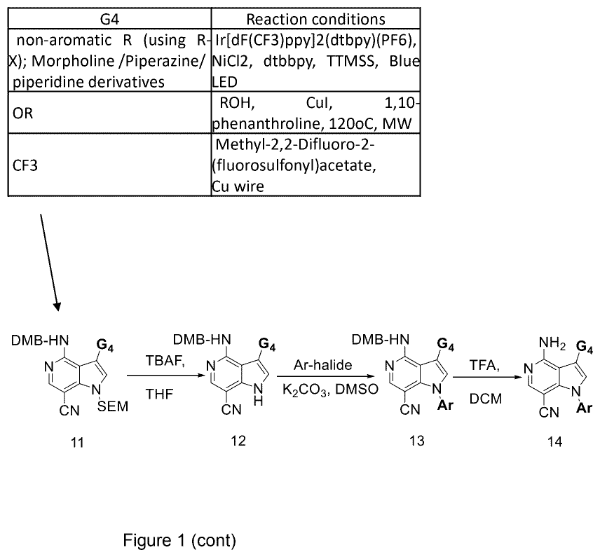

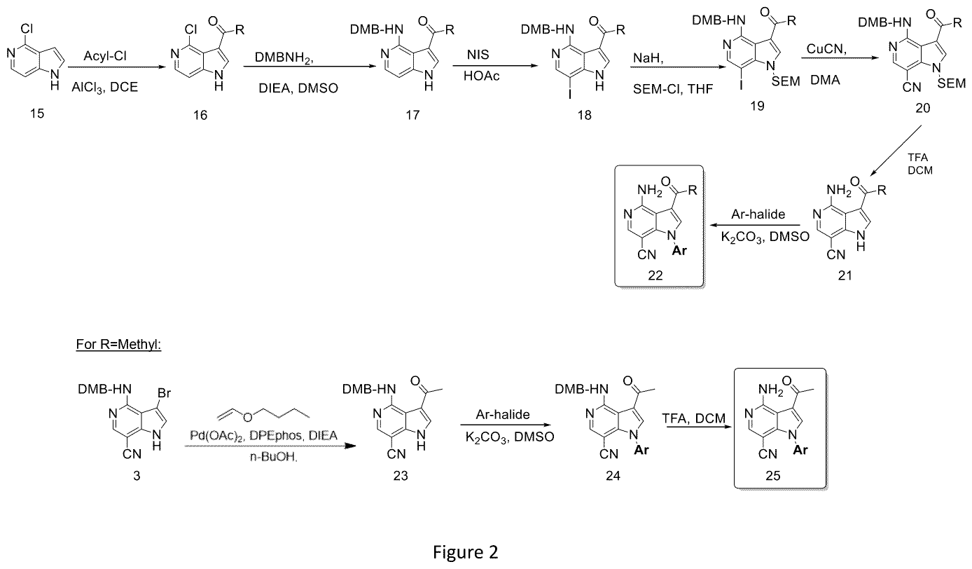

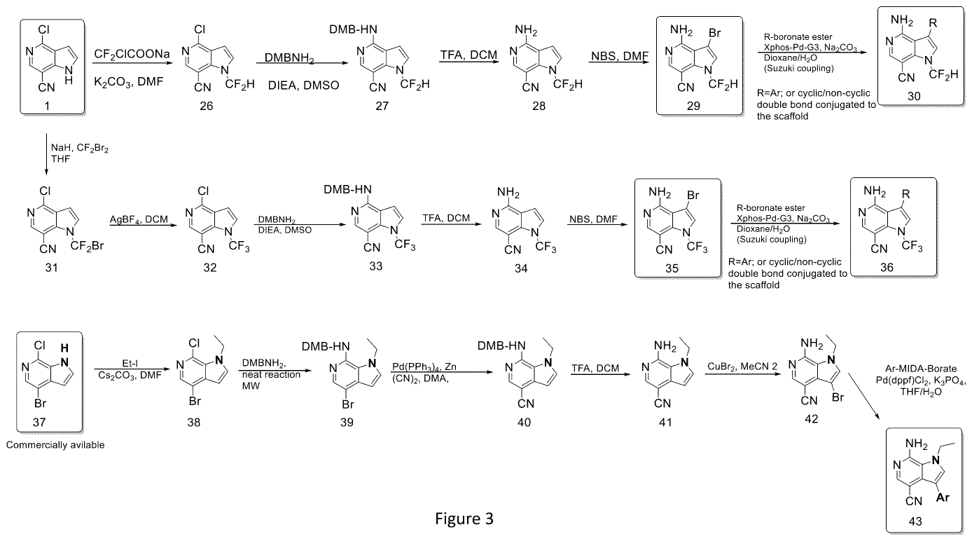

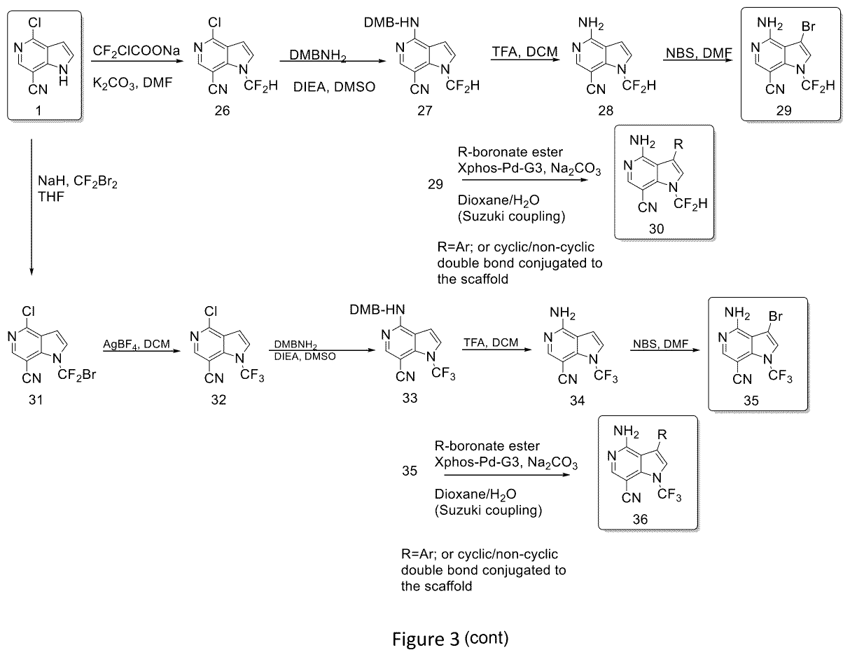

General synthetic schemes are presented in FIGS. 1-3.

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A compound represented by Formula IA: 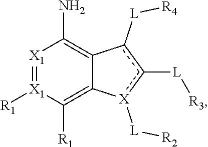
or by Formula IB: 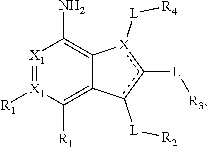
wherein: [symbol]
represents a single or a double bond;
each L is absent or independently comprises an optionally substituted C₁-C₆ alkyl, N, NH, S, and O, —S(O)₂—, —C(O)—, —(C₁-C₆)—O—(C₁-C₆)₀₋₁—, —(C₁-C₆)—S—(C₁-C₆)₀₋₁—, —(C₁-C₆)—NH—(C₁-C₆)₀₋₁—, or any combination thereof;
each X₁ is independently selected from the group consisting of CH, C, or N, and at least one X₁ is N;
X comprises S, O, N or NH;
each R₁ independently represents hydrogen, or a substituent comprising halogen, —NO₂, —CN, —OH, —(C═O)NH₂, HCONH—, oxo, carbonyl, amino, imino, thioxo, —OR, —(C═O)NH₂, —CO₂R, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, C₁-C₆ haloalkyl, optionally substituted C₁-C₆ alkyl, —NH₂, —NR₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), amino(C₁-C₆ alkyl), C₁-C₆ mercaptoalkyl, —CONH(C₁-C₆ alkyl), —CON(C₁-C₆ alkyl)₂, —CO₂H, —CO₂R, —OCOR, —OC(═O)OR, —OC(═O)NR, —OC(═S)OR, —OC(═S)NR, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; wherein at least one R₁ represents the substituent;
any of R₂, R₃ and R₄ is independently H or represents a substituent comprising halogen, —NO₂, —CN, —OH, —OR, —SR, —(C═O)NH₂, —CONR₂, —CO₂R, —SO₂R, oxo, carbonyl, amino, C₁-C₆ mercaptoalkyl, optionally substituted C₁-C₆ alkyl, optionally substituted C₁-C₆ heteroalkyl, imino, thioxo, carbonyl, aminoalkyl, fluoroalkyl, optionally substituted alkyl-aryl/heteroaryl, —O—C₁-C₆ haloalkyl, —O—C(R)₂—C₁-C₆ haloalkyl, —NR₂, -M-W, an optionally substituted C₃-C₈ saturated/unsaturated heterocyclyl, optionally substituted C₃-C₈ saturated/unsaturated cycloalkyl, optionally substituted C₂-C₆ alkenyl, optionally substituted C₂-C₆ alkynyl, —C(═O)—R, —S—C₁-C₆ haloalkyl, —NH—C₁-C₆ haloalkyl, —NR₂, —NH(C₁-C₆ alkyl), hydroxy(C₁-C₆ alkyl), C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof, or any of R₂, R₃ and R₄ is absent;
wherein at least one of R₂ and R₃ is not H or absent;
wherein M is selected from —O—, —S—, —NH—, —C(═O)—, —CONR—, —CNNR—, —CSNR—, —CONH—O—, —CONH—NH—, —NHCO—, —NHCSR—, —NHCN—, —NC(═O)O—, —NC(═O)N—, —NC(═S)O—, —NC(═S)N—, —SO₂—, —SO—, —SO₂O—, —SO₂NR—, —NHNR—, —CO₂—, —OCO—, —OC(═O)O—, —OC(═O)N—, —OC(═S)O— and —OC(═S)N—;
wherein W represents an optionally substituted C₃-C₈ cycloalkyl optionally substituted C₃-C₈ heterocyclyl optionally substituted aryl, optionally substituted alkyl-aryl/heteroaryl, or optionally substituted heteroaryl;
wherein each R independently represents hydrogen, or is selected from the group comprising optionally substituted C₁-C₁₀ alkyl, optionally substituted C1-C10 alkenyl/alkynyl, hydroxy, amino, —NH₂, —NR₂, —NH(C₁-C₆ alkyl), —N(C₁-C₆ alkyl)₂, C₁-C₆ alkoxy, —OR, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR₂, C₁-C₆ alkyl-SR, optionally substituted C₃-C₁₀ cycloalkyl, optionally substituted C₁-C₁₀ alkaryl (e.g. benzyl), optionally substituted C₁-C₁₀ alkyl-C₃-C₁₀ cycloalkyl, optionally substituted C₃-C₁₀ heterocyclyl, optionally substituted heteroaryl, optionally substituted aryl, or a combination thereof; or a salt thereof, wherein said compound comprises any tautomer, any isotope, or any salt thereof.

2. The compound of claim 1, wherein said compound is represented by Formula III: 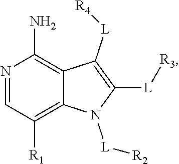
or by Formula IIIA 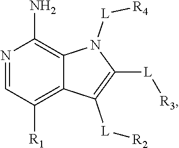
wherein R₁ is selected from halo, cyano, and formamidyl; and
wherein R₂ represents H or a substituent comprising C₁-C₆ haloalkyl, C₁-C₆ fluoroalkyl, —NR₂, C₁-C₆ alkyl-NR₂, C₁-C₆ alkyl-OR, C₁-C₆ alkyl-CONR₂, C₁-C₆ alkyl-COOR, C₁-C₆ alkyl-CNNR₂, C₁-C₆ alkyl-CSNR₂, optionally substituted C₅ or C₆ heteroaryl; and if R2 is H then R3 is not H.

3. The compound of claim 1, wherein said compound is represented by or comprises Formula IV: 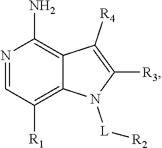
wherein R₁ is selected is selected from halo (e.g. fluoro), cyano, and formamidyl; wherein R₂ represents H or a substituent comprising C₁-C₆ fluoroalkyl, —NR₂ or optionally substituted C₅ or C₆ heteroaryl; and if R2 is H then R3 is not H.

4. The compound of claim 1, wherein R3 is H, and R₂ represents any of: 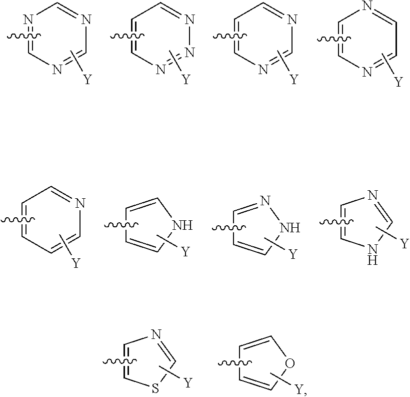
wherein Y is hydrogen or represents one or more substituents, each independently selected from halo, C₁-C₆ fluoroalkyl, —NO₂, —NH₂, —O(C₁-C₆ alkyl), —OMe, —NH(C₁-C₆ alkyl), —N(C₁-C₆ alkyl)₂, —CN, —OH, —(C═O)NH₂, —CONR₂, —CNNR₂, —CSNR₂, —CONH—OH, —CONH—NH₂, —NHCOR, —NHCSR, —NHCNR, —NC(═O)OR, —NC(═O)NR, —NC(═S)OR, —NC(═S)NR, —SO₂R, —SOR, —SR, —SO₂OR, —SO₂N(R)₂, —NHNR₂, —NNR, —(C₁-C₆)—OH, or a combination thereof.

5. The compound of claim 1, wherein R₃ is selected from halogen, —NO₂, —CN, —OH, —OR, —(C═O)NH₂, —CONR₂, —SR, —CO₂R, —SO₂R, optionally substituted C₁-C₆ alkyl, amino, oxo, imino, thioxo, carbonyl, aminoalkyl, —NH₂, —NH(C₁-C₆ alkyl), hydroxy(C₁-C₆ alkyl), C₁-C₆ haloalkyl, C₁-C₆ mercaptoalkyl, C₁-C₆ haloalkyl, optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof.

6. The compound of claim 1, wherein said compound is represented by Formula V: 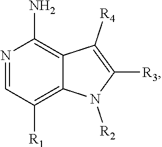
or by Formula VA: 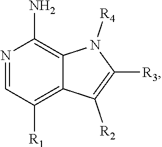
wherein:
R₁ is selected from halo, cyano, and formamidyl;
R₃ is H or is selected from (i) halogen, —NO₂, —CN, —OH, —OR, —(C═O)NH₂, —CONR₂, —SR, —CO₂R, —SO₂R, optionally substituted C₁-C₆ alkyl, amino, oxo, imino, thioxo, carbonyl, aminoalkyl, —NH₂, —NH(C₁-C₆ alkyl), hydroxy(C₁-C₆ alkyl), C₁-C₆ haloalkyl, C₁-C₆ mercaptoalkyl, C₁-C₆ haloalkyl and (ii) optionally substituted C₃-C₈ cycloalkyl, optionally substituted C₃-C₈ heterocyclyl, optionally substituted heteroaryl, optionally substituted aryl, optionally substituted cycloalkyl, optionally substituted heterocyclyl, optionally substituted bicyclic heteroaryl, optionally substituted bicyclic aryl, optionally substituted bicyclic heterocyclyl, optionally substituted bicyclic cycloalkyl, or a combination thereof; if R₃ is H or (i) then R₂ is selected from C₁-C₆ fluoroalkyl and a heteroaryl optionally substituted by one or more substituents, optionally wherein the substituents are interconnected so as to form a 5-, or 6-membered aliphatic, unsaturated or aromatic ring;
if R₃ or R₄ is H then R₂ is not H;
if R₃ is (ii) then R₂ is C₁-C₆ fluoroalkyl.

7. The compound of claim 6, wherein R₃ is H, R₂ is selected from C₁-C₆ fluoroalkyl and a heteroaromatic ring optionally substituted by one or more substituents, optionally wherein the substituents are interconnected so as to form a 5-, or 6-membered aliphatic, unsaturated or aromatic ring.

8. The compound of claim 1, wherein R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

9. The compound of claim 1, wherein R₂ is a C1-C3 fluoroalkyl, or a 5-6 membered heteroaromatic ring substituted by one or more substituent selected from —F and C1-C3 fluoroalkyl.

10. The compound of claim 6, represented by Formula VB: 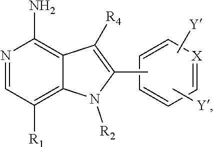
wherein X is N or CH; wherein each Y′ is independently hydrogen or one or more substituent selected from optionally substituted C₁-C₆ alkyl, —OR, —NR₂, —O(C₁-C₆ alkyl), —OMe, —NH(C₁-C₆ alkyl), —N(C₁-C₆ alkyl)₂, —CN, —OH, —NHCOR, —NHNR₂, —NNR, C₁-C₆ haloalkyl, —(C₁-C₆)OH, or wherein both Y′ are interconnected so as to form a ring; and wherein R₂ is selected from H and C₁-C₆ fluoroalkyl; and wherein R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

11. The compound of claim 1, represented by Formula VI: 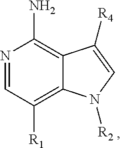
wherein R₁ is selected from cyano and formamidyl;
R₂ is selected from C₁-C₆ haloalkyl, optionally substituted alkyl-aryl/heteroaryl, and a 5-6 membered heteroaromatic ring optionally substituted by at least one of —F and C1-C3 fluoroalkyl;
R₄ is H or is selected from halogen; —OR; —O—C₁-C₆ haloalkyl; —O—C(R)₂—C₁-C₆ haloalkyl; C₁-C₆ haloalkyl; optionally substituted C₁-C₆ alkyl; -M-W; an optionally substituted C₃-C₈ saturated/unsaturated heterocyclyl; optionally substituted C₃-C₈ saturated/unsaturated cycloalkyl; optionally substituted C₂-C₆ alkenyl; —CN; optionally substituted C₂-C₆ alkynyl; —C(═O)—R; —SR; —S—C₁-C₆ haloalkyl; —NH—C₁-C₆ haloalkyl; —NO₂; —OH; —(C═O)NH₂; —CONR₂; —CO₂R; —SO₂R; —NH₂; —NR₂; —NH(C₁-C₆ alkyl); hydroxy(C₁-C₆ alkyl); optionally substituted heteroaryl; optionally substituted aryl; optionally substituted heterocyclyl; optionally substituted bicyclic heteroaryl; optionally substituted bicyclic aryl; optionally substituted bicyclic heterocyclyl; and optionally substituted bicyclic cycloalkyl.

12. The compound of claim 11, wherein R₂ is selected from —CH2F and a 5-6 membered heteroaromatic ring substituted by at least one of —F and C1-C3 fluoroalkyl; wherein R₂ is selected from —CH2F and pyridine substituted by at least one of —F and C1-C3 fluoroalkyl; wherein R₄ is selected from fluoroalkyl, halo, —OR, —C(═O)—R, substituted C₃-C₆ cycloalkyl, substituted C₃-C₆ heterocyclyl comprising an unsaturated bond and a substituted aryl; and wherein said substituted comprises at least one substituent selected from haloalkyl, C₁-C₆ haloalkyl, alkoxy, C₁-C₆ hydroxyalkyl, oxo, optionally substituted C₁-C₆ alkyl and —C(═O)—R.

13. (canceled)

14. (canceled)

15. The compound of claim 1, wherein said compound is or comprises any one of compounds of Table 1 or FIG. 4, including any salt, any isotope or any tautomer thereof.

16. The compound of claim 1, wherein said compound is stable in an aqueous solution for at least 1 hour.

17. A pharmaceutical composition, comprising the compound of claim 1 and a pharmaceutically acceptable carrier.

18. (canceled)

19. (canceled)

20. A method for preventing or treating a disease or a disorder or ameliorating a condition associated with an activity of PKR in a subject, comprising administering to the subject the pharmaceutical composition of claim 17, thereby (i) preventing or treating a disease or a disorder; or (ii) ameliorating said condition associated with an abnormal expression and/or activation of said PKR in a subject.

21. The method of claim 20, wherein said disease or said disorder comprises a neurodegenerative disorder, a neuroinflammatory disorder, a metabolic disease, a cardiovascular disease, a proliferative disease, or any combination thereof.

22. (canceled)

23. (canceled)

24. The method of claim 20, wherein said administering is by an oral administration, a topical administration, nasal administration, sublingual administration, buccal administration, a systemic administration, or any combination thereof.

25. The method of claim 20, wherein said ameliorating comprises (i) improving cognitive function, or (ii) inhibiting cognitive dysfunction of the subject; and wherein said subject comprises a human subject, an animal subject or both.

26. (canceled)

27. A method for enhancing or improving cognition in a subject in need thereof, comprising administering to the subject the pharmaceutical composition of claim 17, thereby enhancing or improving cognition in a subject in need thereof.
